(19) United States
(12) Patent Application Publication
(10) Pub. No.: US 2025/0297548 A1
(43) Pub. Date: Sep. 25, 2025

(54) DETERMINING HYDRAULIC FRACTURING TREATMENT METHODS FOR WELLS IN CARBONATE RESERVOIRS

(71) Applicant: Saudi Arabian Oil Company, Dhahran (SA)

(72) Inventors: Kaiming Xia, Dhahran (SA); Weihua Wang, Dhahran (SA); Rasim Serdar Rodoplu, Dhahran (SA)

(21) Appl. No.: 18/609,562

(22) Filed: Mar. 19, 2024

(51) Int. Cl. E21B 49/02; E21B 43/26; E21B 43/267; E21B 43/27; G01N 33/24; G01V 20/00

(52) U.S. Cl. CPC E21B 49/02; E21B 43/267; E21B 43/27; G01N 33/24; G01V 20/00; E21B 43/2607; E21B 2200/20

(57) ABSTRACT

Techniques for stimulating a formation surrounding a wellbore include determining a rock type of the formation along the well trajectory in landing zone in terms of measured depth. A depth-specific acid fracture conductivity parameter of the formation and a depth-specific fracture conductivity declining parameter of the formation are determined. One or more depth intervals requiring fracturing are determined based on the rock type of the formation, the acid fracture conductivity parameter of the formation, and the fracture conductivity declining parameter of the formation. A fracturing method is determined for each depth interval of the one or more depth intervals based on the acid fracture conductivity parameter of the formation and the fracture conductivity declining parameter of the formation. A corresponding pump schedule of the fracturing method is determined, and a fluid of the pump schedule is pumped into the wellbore to fracture or stimulate the formation.

### TECHNICAL FIELD

The present disclosure describes systems and methods for hydraulic fracturing treatment in deep & tight carbonate reservoirs determined based on rock typing, petrophysical & geomechanical properties of the formation.

### BACKGROUND

Development of deep and tight carbonate reservoirs is critical because of their vast distribution and hydrocarbon accumulation. Hydraulic fracturing is important for these reservoirs because formation closure pressures are high, and the flow of oil and gas through the formation can be limited depending on rock lithology, petrophysical & geomechanical properties of the formation. Deciding on a particular fracturing method in carbonate reservoirs is nontrivial. Using the wrong fracturing method can lead to reduced or non-existent oil and gas flow through the formation.

### SUMMARY

Challenges and uncertainties arise in the commonly used acid fracturing for deep and tight carbonate gas reservoirs. This disclosure provides a method for improving stimulating carbonate reservoirs, which evaluates the rock quality, calculates the breakdown pressure envelope, optimal perforation direction, evaluates the potential fracture conductivity, and determines a suggested stimulation method using either acid fracturing or proppant fracturing with verification by a fracturing simulation.

The systems and methods fracture the formation using acid fracturing when an acid fracture conductivity parameter is high and a fracture conductivity declining parameter is low. The systems and methods fracture the formation using proppant fracturing when the acid fracture conductivity parameter is low and the fracture conductivity declining parameter is high. A computing system determines the acid fracture conductivity parameter and the fracture conductivity declining parameter based on wellbore data, modeling data, and physical testing.

High acid fracture conductivity parameters can indicate better acid fracturing performance while a lower acid fracture conductivity parameter can indicate that the formation is not suitable for acid fracturing. High fracture conductivity declining parameters mean that the uneven fracture surface may be crushed under the formation closure pressure and maintaining the acid fracture conductivity can be difficult.

Acid fracturing has both advantages and disadvantages relative to proppant fracturing (which may also be used on carbonates). Among the advantages of acid fracturing are that the operation can be carried out in the field with no risk of screen out. Fracturing treatments carrying propping agents sometimes screen out. Such failures do not occur with acid fracturing. In addition, acid fracturing can often be less expensive than proppant fracturing due to less equipment use (for example, no proppant handling equipment or blender is required with acid fracturing). Finally, acid fracturing can be designed with somewhat less sophisticated tools than proppant fracturing and thus can often be accomplished with greater certainty.

In many cases, the choice between proppant fracturing and acid fracturing is made after a thorough evaluation of the potential and limitations of each treatment for the specific job intended. This disclosure provides a method and system for fracturing deep and tight carbonate gas reservoirs and can improve the final outcome from the stimulation treatments.

The success of hydraulic fracturing treatment has been important for producing deep and tight carbonate reservoirs. Acid fracturing has been generally used to stimulate wells landed in carbonate formations. Compared to proppant hydraulic fracturing, acid fracturing generates fracture conductivity through acid etching the fracture surface, in which proppant is not used. However, the fracture conductivity is more difficult to maintain for longer durations especially in high formation closure pressure environments. Experiments showed that propped hydraulic fractures would retain a great conductivity under the same formation closure stress compared to the acid fractures. Besides this, for wells landing in deep and tight carbonate reservoirs with high formation closure pressure, mineralogy plays a significant role during acid fracturing. In addition, fracture initiation also requires a high breakdown pressure. In this situation, locating the ideal perforation locations along the wellbore is an important priority for hydraulic fracturing designs. Considering all these factors together, this disclosure provides a method for determining the fracturing treatment type for wells in deep and tight carbonate gas reservoirs.

The systems and methods of this disclosure calculate rock typing and reservoir quality, breakdown pressure envelope, optimal perforation directions, acid fracture conductivity index, identifying perforation locations, and finally selecting the appropriate fracturing method (e.g., acid fracturing, or proppant fracturing) along the landing part of a given well trajectory. This can ensure fractures are initiated at a relatively lower breakdown pressure but also increase the chances that the fractures propagate into gas bearing good rock quality areas, consequently resulting with good and sustainable fracture conductivity.

The workflow described in this disclosure identifies the ideal perforation locations where rock types are good and also initiate the fractures with a lower required breakdown pressure. In the situation where sweet spot areas need high breakdown pressure, oriented perforation can be used to increase fracture initiation success rate. After this is done, the workflow checks the acid fracture conductivity parameter and fracture conductivity declining parameter, which lead to a determination of whether to use acid fracturing or proppant fracturing.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is diagram of an example wellbore system.

FIGS. 2A-2D are images of example rock lithologies for carbonate reservoirs.

FIG. 3 is a plot of rock typing results as a function of depth of a wellbore.

FIG. 4 is a plot of in-situ stresses acting on a wellbore.

FIG. 5A is a plot of wellbore and perforation coordinate systems.

FIG. 5B is a plot showing the maximum horizontal stress acting on the wellbore.

FIG. 6 is a plot of a wellbore with three perforations and coordinate systems.

FIG. 7 is a view showing the stresses acting on a wellbore and perforation tunnels.

FIG. 8 is a schematic of stress superposition of wellbore stresses.

FIG. 9 is a plot of stresses acting on a wellbore.

FIG. 10 is an exploded view of radial stresses acting on the components of the wellbore.

FIG. 11 is a view of a wellbore with perforation tunnels.

FIG. 12 is a schematic of stress superposition of wellbore stresses.

FIGS. 13A-13C are diagrams an initial fracture, an acid-etched fracture, and propped fracture, respectively.

FIG. 14 is a flowchart of an example method for determining rock typing, sweet spot identification, breakdown pressure, optimal perforation locations & directions, acid fracture conductivity parameter, fracture declining parameter, and suggested fracturing methods for each perforation location.

FIGS. 15A-15C are diagrams of an example wellbore system where both proppant fracturing and acid fracturing are used in a single well.

FIG. 16 is a log plot indicating rock types that vary as a function of trajectory of a wellbore.

FIG. 17 is a log plot indicating breakdown pressures and perforation angles that vary as a function of trajectory of a wellbore.

FIG. 18 is a log plot indicating acid fracture conductivity parameters and fracture conductivity declining parameters that vary as a function of trajectory of a wellbore.

FIG. 19 is a log plot indicating suggested perforation locations and a suggested fracturing method for each perforation location.

FIG. 20 is a flowchart of an example method for determining rock typing, acid fracture conductivity parameter, fracture declining parameter, perforation locations, and a suggested fracturing method for each perforation location.

FIG. 21 is a schematic of an example controller for determining perforation locations and a suggested fracturing method for each perforation location.

### DETAILED DESCRIPTION

The systems and methods described in this disclosure relate to hydraulic fracturing treatment in deep & tight carbonate reservoirs determined based on rock typing, petrophysical & geomechanical properties of the formation. The systems and methods recommend fracturing the formation using acid fracturing when an acid fracture conductivity parameter is high and a fracture conductivity declining parameter is low. The systems and methods recommend fracturing the formation using proppant fracturing when the acid fracture conductivity parameter is low and the fracture conductivity declining parameter is high. A computing system determines the acid fracture conductivity parameter and the fracture conductivity declining parameter based on wellbore data, modeling data, and physical testing. In some examples, the systems and method fracture the formation based on the recommended fracturing technique.

### Wellbore System

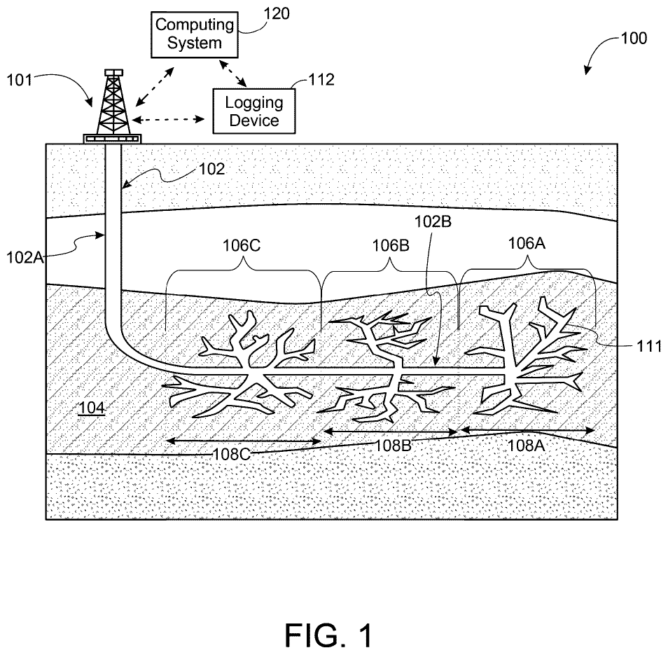

FIG. 1 is a diagram of a wellbore system 100. The wellbore system 100 includes a well 101 with a wellbore 102 that has been drilled into one or more layers of a formation 104 (e.g., by a drilling rig). The wellbore 102 includes a substantially vertical section 102A and a substantially horizontal section 102B. In some examples, the wellbore 102 is drilled such that the horizontal section 102B lies in, or near, a carbonate reservoir. Hydrocarbons (e.g., oil and gas) are extracted from the reservoir to a ground surface of the well 101.

Wellbore system 100 includes three perforation stages 106A, 106B, and 106C. Each perforation stage 106A-C is associated with a depth interval 108A, 108B, 108C, respectively, within the wellbore 102. For example, perforation stage 106A is the deepest in the well, followed by perforation stage 106B, followed by perforation stage 106C being the shallowest. Depth can represent the true vertical depth in the wellbore 102 or it can represent the measured depth in the wellbore 102 (e.g., as measured by a downhole logging tool 112). In the example shown, depth represents measured depth in the wellbore 102. While illustrated as three perforation stages, some wells have more than three (e.g., 4-10) perforation stages.

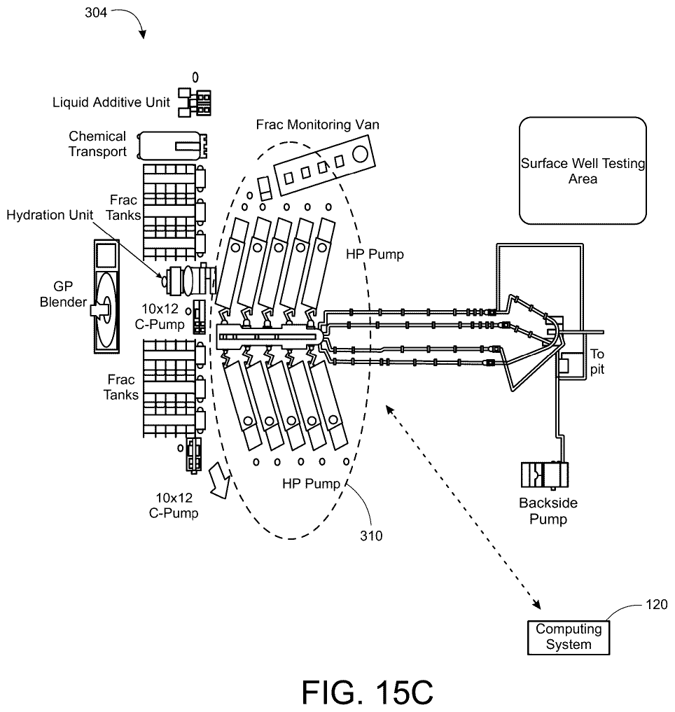

Each perforation stage 106A-C includes one or more perforation tunnels connecting to fractures 111 extending from the wellbore 102 into the formation 104. In some examples, the perforation tunnels (see FIG. 15 which shows example perforation tunnels 110) are formed by charged explosives. In some examples, the perforation tunnels are formed by using hydra-jetting to jetting high speed fluid to perforate the formation. In some cases, this process is referred to as a pad stage of the well 101. In some examples, acid fracturing and proppant fracturing is used to expand the fractures 110 or keep them open to avoid a premature closure of the perforation tunnels.

Determining the locations of the perforation stages in the wellbore 102 is an important aspect of well placement and design. In some cases, it is preferable to include as many perforation stages 106A-C and perforation tunnels 110 as possible to increase the channel size and number of flow paths for hydrocarbons to flow from the formation 104, to the wellbore 102, and to the ground surface of the well 101. However, determining where to locate the perforation stages and tunnels is nontrivial. For example, formation lithology, porosity, permeability, and breakdown pressure can vary significantly with depth.

### Rock Typing

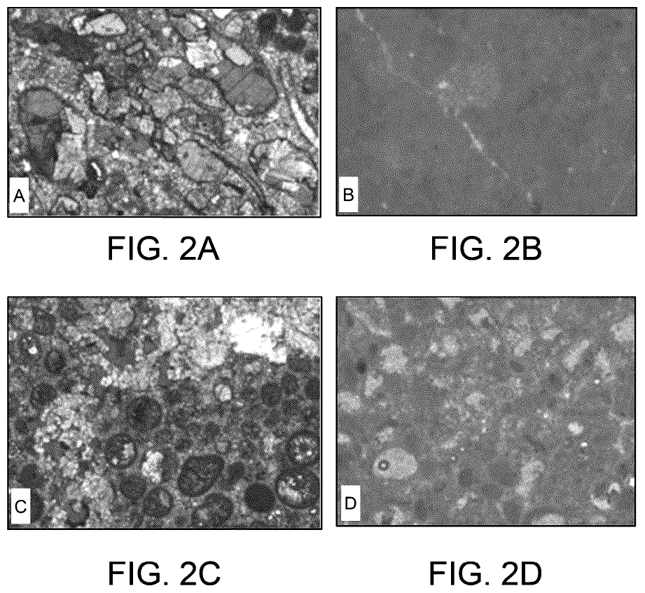

FIGS. 2A-2D are images of example rock lithologies for carbonate reservoirs. Tight grain supported limestone (FIG. 2A) and tight mud supported dolomite (FIG. 2B) have a low porosity and are more difficult to flow hydrocarbons through them. Porous grain supported limestone (FIG. 2C) and porous grain supported dolomite (FIG. 2D) have a higher porosity (e.g., moldic porosity), a low permeability, and are easier to flow hydrocarbons through them. Hydraulic fracturing can be used to connect the moldic pores. Dolomitization is common in carbonate reservoirs and can reduce or increase carbonate total porosity. Porosity and lithology are important factors for hydraulic fracturing. For relatively higher porosity and low permeability carbonate (e.g., FIGS. 2C and 2D), hydraulic fracturing is useful to connect the moldic pores to define a flow path through the rock.

In some examples, the logging device 112 (or wireline log) is lowered into the wellbore 102 to acquire depth-specific data about the formation 104 surrounding the wellbore 102. The term “depth-specific” data means that the data can vary along a well trajectory of the well in the landing zone and can be expressed with respect to the well measured depth (which as noted above may not correspond to the true vertical depth). In some cases, the logging device 112 measures material properties of the formation (e.g., an effective porosity of the formation 104, a permeability of the formation 104, a volume of calcite in the formation 104, a volume of dolomite in the formation 104, and/or a volume of anhydrite in the formation 104). In some cases, the logging device 112 measures geometric properties of the formation (e.g., depth, wellbore diameter, etc.). In some examples, the logging device 112 measures depth-specific data of the rock lithology of the formation 104 after an initial petrophysical evaluation of the formation 104 (e.g., after the wellbore 102 has been drilled).

In some examples, the logging device 112 comprises sonic log that emits sound waves through the formation and measure a reflected portion of the sound waves to measure rock properties of the formation.

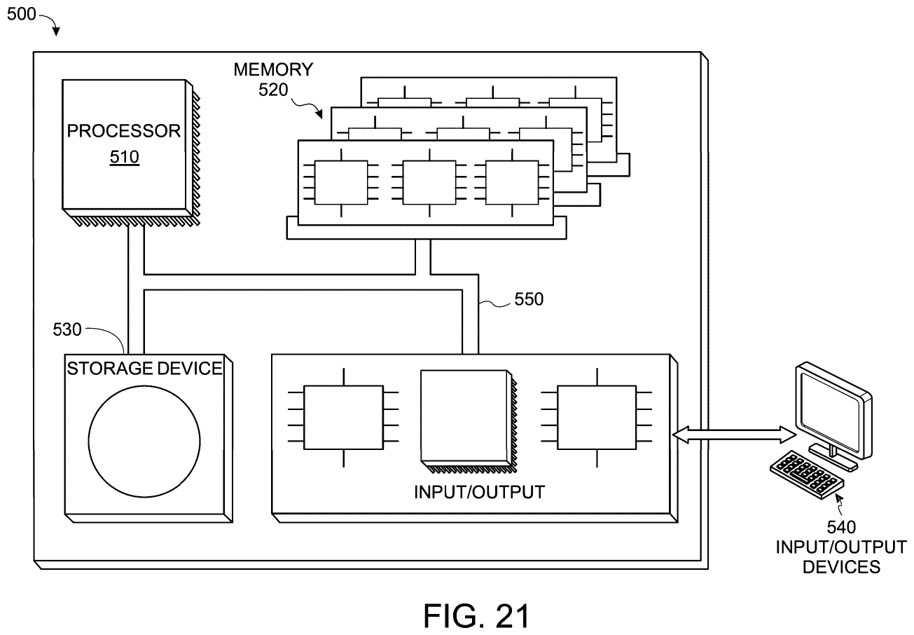

In some examples, the logging device 112 is in communication with a computing system 120 and transmits the measured depth-specific data of the formation 104 to the computing system 120. In some examples, the computing system 120 is or includes a computer with a processor configured to perform or control one or more operations of the wellbore system 100. In some examples, the computing system 120 is or includes the controller 500 described with reference to FIG. 21. In some examples, diagnostic injection tests are performed before main treatments and the computing system 120 compares an estimated breakdown/closure pressures and updates the calculations for a treatment type decision.

The computing system 120 defines a plurality of rock types of the formation 104 based on the depth-specific data from the logging device 112. For example, as shown in Table 1 below, six different rock types are established for the wellbore system 100. Each rock type has a unique combination of effective porosity and rock lithology.

TABLE 1
Carbonate log-based rock typing criteria.

| Rock Type | Effective Porosity | Lithology |
|---|---|---|
| 6 | >5% | Dolomite > Calcite |
| 5 | >5% | Calcite > Dolomite |
| 4 | 3%<=, =>5% | Dolomite > Calcite |
| 3 | 3%<=, =>5% | Calcite > Dolomite |
| 2 | <3% | Calcite + Dolomite > Anhydrite |
| 1 | <3% | Anhydrite > Calcite + Dolomite |

For example, the computing system 120 identifies a rock as type 6 when the effective porosity is greater than 5% and the volume of dolomite is greater than a volume of calcite. The computing system 120 identifies a rock as type 5 when the effective porosity is greater than 5% and the volume of calcite is greater than a volume of dolomite. In Table 1, higher rock types (e.g., 6, 5, etc.) indicate better rock quality and are better for hydraulic fracturing and production than lower rock types (e.g., 1, 2, etc.). While six rock types are represented in Table 1, in some examples, more than six (e.g., 7-10) different rock types are used by the computing system 120, and in some examples, less than six (e.g., 2-5) different rock types are used. While Table 1 specifies specific criteria, other criteria can be used. In some examples, effective porosities above 10% are better than effective porosities below 10%. In some examples, more calcite is preferred over less calcite. In some examples, a higher percentage of calcite is preferred over a lower percentage of dolomite.

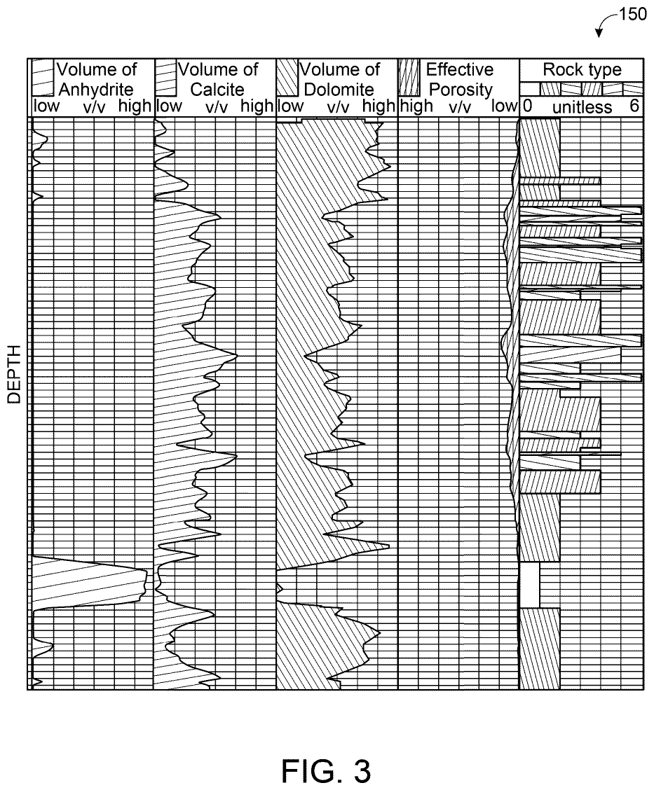

FIG. 3 is a log plot 150 of depth-specific rock typing results for the example wellbore 100 as determined by the computing system 120. Depth increases vertically from the top of the log plot 150 (the shallowest) to the bottom of the log plot 150 (the deepest). From the left, track 1 indicates the volume of anhydrite in the formation 104 as measured by the logging device 112, track 2 indicates the volume of calcite in the formation 104 as measured by the logging device 112, track 3 indicates the volume of dolomite in the formation 104 as measured by the logging device 112, track 4 indicates effective porosity of the formation 104 as measured by the logging device 112, and track 5 indicates the determined rock type as a function of measured depth in the wellbore 102. In this example, some depths are determined to have rock types of 1 or 2 which are less ideal for hydraulic fracturing and production and some depths are determined to have rock types of 4, 5, or 6 which are better for hydraulic fracturing and production.

### Breakdown Pressure and Perforation Direction

It is preferable to also estimate the breakdown pressure of the formation 104 as accurately as possible. Breakdown pressure is an important factor because fracturing cannot go as planned if the pressure of the pad fluid (or explosives) is not greater than the breakdown pressures in the formation 104 for a given perforation stage. In extreme cases, this can lead to skipping one or more stages and can reduce the production of a well.

For a specific lithology and material properties of the formation, the required breakdown pressure is dependent on perforation direction (e.g., vertical, horizontal, etc.). U.S. Pat. No. 11,255,184 (Attorney Docket No. 38136-1274001), U.S. Pat. No. 11,391,135 (Attorney Docket No. 38136-1349001), U.S. Publication No. 2022/0259960 (Attorney Docket No. 38136-1352001), and U.S. Publication No. 2023/0064121 (Attorney Docket No. 38136-1576001), the disclosures of which are incorporated by reference in their entirety, disclose analytical formulations and numerical techniques to calculate required breakdown pressures and the corresponding perforation orientation directions for arbitrary well trajectories based on logging data and laboratory testing of core plugs.

The computing system 120 determines the required breakdown pressures and corresponding perforation orientation directions as a function of depth in the wellbore 102 based on the depth-specific data from the logging device 112 and laboratory testing of core plugs that have been extracted from the formation 104.

In some implementations, the computing system 120 identifies in-situ stresses for a wellbore formed through a formation. In some aspects, the logging tool 112 may derive or generate an image log of the subsurface formation 104, from which the maximum principal stress angle of the subsurface formation 104 can be obtained. From the maximum principal stress angle and borehole image, a maximum principal stress of the subsurface formation 104 can be estimated and in-situ stresses may be calibrated and finally determined.

In some aspects, the computing system 120 calculate the in-situ stresses according to a number of parameters. For example, such parameters may include wellbore True Vertical Depth (TVD), azimuth angle, and deviation angle and the image log. The parameters may also include the mechanical properties of, for example, a casing of the wellbore 102, a cement between the casing and the formation 104, and the subsurface formation 104 itself (for example, the tensile strength of the subsurface formation 104).

For example, an in-situ stress field of the subsurface formation 104 exists in the far field and takes the form as follows:

$${σ}_{i n} = ( \begin{matrix}{σ}_{xx} & {τ}_{xy} & {τ}_{xz} \\ {τ}_{yx} & {σ}_{yy} & {τ}_{yz} \\ {τ}_{zx} & {τ}_{zy} & {σ}_{zz}\end{matrix} ) or {σ}_{Pr} = ( \begin{matrix}{σ}_{Hmax} & 0 & 0 \\ 0 & {σ}_{Hmin} & 0 \\ 0 & 0 & {σ}_{V}\end{matrix} ) . \qquad ( 1 )$$

In Eq. 1, σ_{Hmax} and σ_{Hmin} are the maximum and minimum horizontal stresses respectively, and Oy is the principal vertical stress component. The dynamic Young's modulus and Poisson's ratio of the subsurface formation 104 can be calculated using, for example, a sonic log from the logging tool 112, then converted to a static modulus based on correlations. The vertical stress S_{V} (total stress) or σ_{V} (effective stress) of the subsurface formation 104 can be reasonably calculated based on, for example, a density log of the logging tool 112, as:

$${S}_{V} = \int ρ dZ , {σ}_{V} = {S}_{V} - α {P}_{0} . \qquad ( 2 )$$

Without considering tectonic stresses, the effective and total minimum horizontal stress can be approximately calculated by:

$${σ}_{Hmin} = \frac{μ}{1 - μ} {σ}_{V} , {S}_{Hmin} = \frac{μ}{1 - μ} ( {σ}_{V} - α {P}_{0} ) + α {P}_{0} . \qquad ( 3 )$$

In Eq. 3, α is the Biot's poroelastic parameter and P₀ is reservoir pressure. The maximum principal stress can be estimated based on, for example, the image log by calibrating the maximum horizontal stress magnitude against a drilling fluid (“mud”) weight and observed breakout and breakdown zone exhibited in the image log data.

In a conventional analysis for a vertical open hole, the maximum principal stress can be obtained based on the breakdown pressure from a leak off test during drilling. Eq. 3 assumes the horizontal strain equal to zero. Under the tectonic regime with given horizontal strains ε_{Hmax} and ε_{Hmin}, the maximum and minimum horizontal stresses can be generally calculated by:

$${S}_{Hmin} = \frac{μ}{1 - μ} ( {σ}_{V} - α {P}_{0} ) + α {P}_{0} + \frac{E}{1 - {μ}^{2}} ( {ε}_{Hmin} + μ {ε}_{Hmax} ) \qquad ( 4 )$$

$${S}_{Hmax} = \frac{μ}{1 - μ} ( {σ}_{V} - α {P}_{0} ) + α {P}_{0} + \frac{E}{1 - {μ}^{2}} ( μ {ε}_{Hmin} + {ε}_{Hmax} ) \qquad ( 5 )$$

Drilling the wellbore 102 in and through the subsurface formation 104 leads to a stress redistribution around the wellbore 102. The wellbore 102 is generally supported by drilling fluid pressure acting on the wellbore wall. Accurately estimating the stresses around the wellbore 102 may be necessary for wellbore stability. Also, it may be helpful to determine the breakdown pressure for hydraulic fracturing design, which directly impacts the selection of casing size, treatment tubing size, wellhead, steel grade, pump schedule, and other equipment.

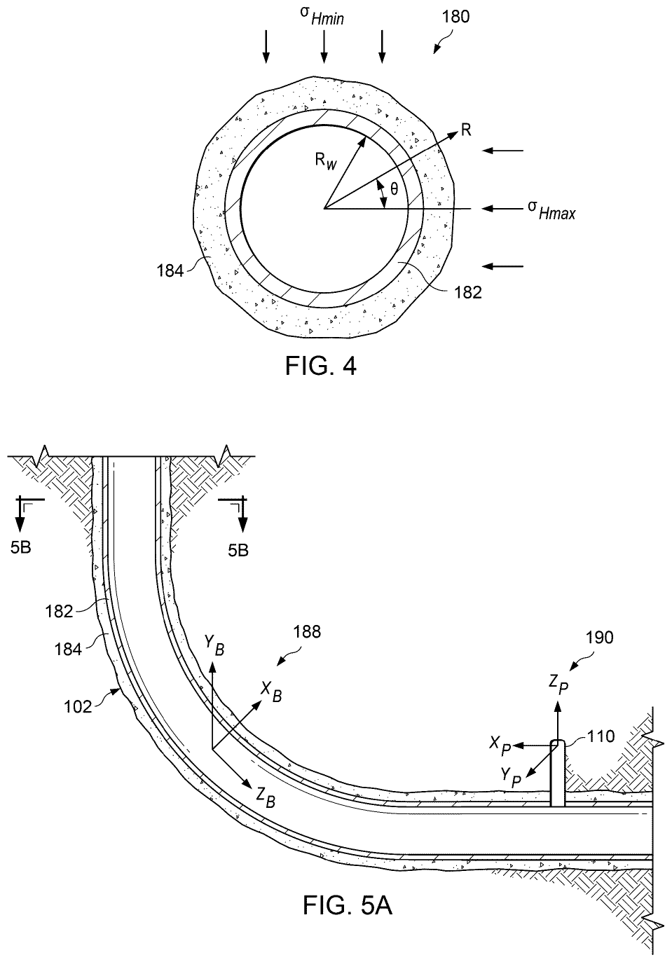

For example, FIG. 4 illustrates a schematic top view cross-section 180 of a cased, vertical wellbore with particular stresses. In cross-section 180, R_{w} represents the wellbore radius, and R represents a radial distance from the concentric center of a casing 182 and cement 184, along with the effective total minimum and maximum horizontal stresses, σ_{Hmax} and σ_{Hmin}.

For a conventional, vertical open-hole wellbore, it is a generally accepted convention that the three far field principal stresses and orientation are known for a conventional vertical, open hole wellbore. The elastic solutions of the effective stresses around wellbore based on plane strain condition are given by:

$${σ}_{r} = \frac{( {σ}_{Hmax} + {σ}_{Hmin} )}{2} ( 1 - \frac{{R}_{w}^{2}}{{R}^{2}} ) + \frac{( {σ}_{Hmax} - {σ}_{Hmin} )}{2} ( 1 - 4 \frac{{R}_{w}^{2}}{{R}^{2}} + 3 \frac{{R}_{w}^{4}}{{R}^{4}} ) + ( {P}_{w} - {P}_{0} ) \frac{{R}_{w}^{2}}{{R}^{2}} , \\ and \qquad ( 6 )$$

$${σ}_{θ} = \frac{( {σ}_{Hmax} + {σ}_{Hmin} )}{2} ( 1 + \frac{{R}_{w}^{2}}{{R}^{2}} ) + \frac{( {σ}_{Hmax} - {σ}_{Hmin} )}{2} ( 1 + 3 \frac{{R}_{w}^{4}}{{R}^{4}} ) cos 2 θ - ( {P}_{w} - {P}_{0} ) \frac{{R}_{w}^{2}}{{R}^{2}} . \qquad ( 7 )$$

In Eqs. 6 and 7, σᵣ is the radial stress acting outwards from the wellbore; σ_{θ} is the hoop stress around the wellbore; θ is the angle from the direction of σₓₓ; P_{w} is the wellbore pressure; and P₀ is the reservoir pressure.

For an open-hole wellbore, limiting this to the wellbore wall with R=R_{w} leads to:

$${σ}_{r} = {P}_{w} - {P}_{0} , \qquad ( 8 )$$

$${σ}_{θ} = ( {σ}_{Hmax} + {σ}_{Hmin} ) - 2 ( {σ}_{Hmax} - {σ}_{Hmin} ) cos 2 θ - ( {P}_{w} - {P}_{0} ) , \\ and \qquad ( 9 )$$

$${τ}_{r θ} = 0. \qquad ( 10 )$$

For the hydraulic fracturing, tensile strength criteria is generally used to direct the fracture propagation trajectory; therefore a fracture propagates at the direction (or orientation angle) of maximum horizontal stress.

The corresponding hoop stress at θ=0 yields:

$${σ}_{θ} = 3 {σ}_{Hmin} - {σ}_{Hmax} - ( {P}_{w} - {P}_{0} ) . \qquad ( 11 )$$

Breakdown pressure is determined based on tensile strength. If the hoop stress turns into tension at wellbore wall and exceeds the material's tensile strength T, the material (in other words, the rock) will fail in tensile mode:

$${σ}_{θ} = - T , \\ and \qquad ( 12 )$$

$${P}_{w} = 3 {σ}_{Hmin} - {σ}_{Hmax} + {P}_{0} + T ( in terms of effective stress ) . \qquad ( 13 )$$

The computing system 120 uses Eq. 13 to predict the required drilling mud weight in the conventional, vertical open-hole example, which can avoid wellbore breakdown issues during drillings. For horizontal wells drilled in the subsurface formations (such as deep and tight reservoirs), the horizontal parts are generally drilled in the minimum horizontal stress direction and thereafter is cased and cemented. After perforating the casing, fluid injection is executed to initiate hydraulic fractures from the perforation towards the maximum horizontal stress direction. In such situations, the computational framework of the present disclosure may calculate the hoop stress around the perforation tunnel for judging whether fracture can be initiated or not, even though the hoop stress with respect to the wellbore is not the main concern. For a cased and cemented wellbore with perforation clusters (such as wellbore 102). The breakdown pressure refers to the bottom hole pressure inside the casing that leads to tensile failure within the area of the wellbore-perforation interface. Therefore, Eq. 13 cannot be directly used to estimate the breakdown pressure for deviated, cased wellbores with clustered perforations for a hydraulic fracturing treatment (such as wellbore 102).

In some implementations, the computing system 120 transforms the in-situ stresses from a global coordinate system to a wellbore coordinate system of a deviated wellbore that includes at least one perforation tunnel. In some aspects, this includes calculating coefficient of pressure transferred, for example, from a hydraulic fracturing fluid to the subsurface formation 104 through the casing 182 and cement 184 as well as pressure loss due to perforation friction.

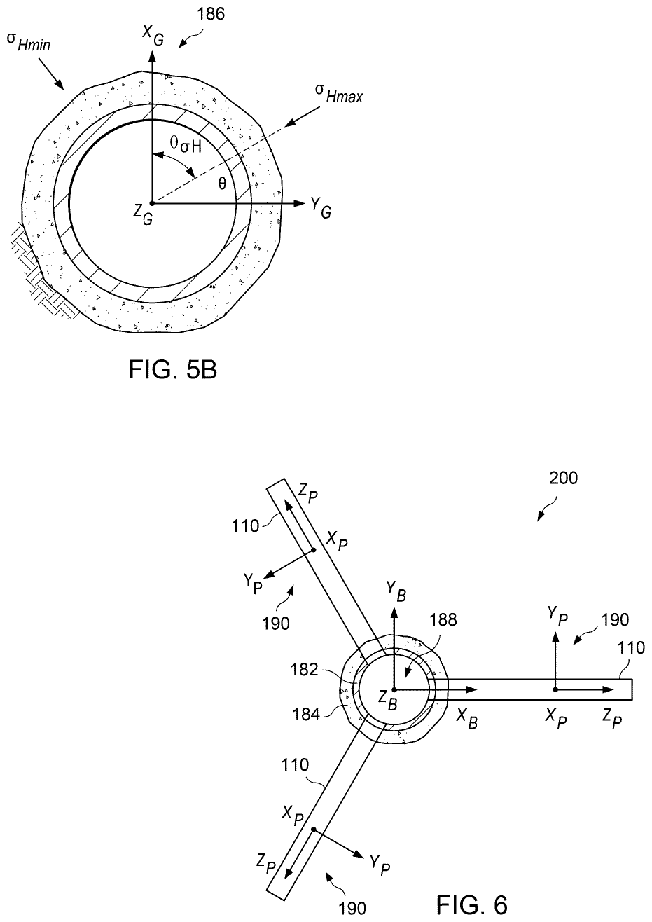

For example, for a deviated wellbore such as wellbore 102, the computing system 120 transforms the in-situ stresses between different coordinate systems. For example, the in-situ stresses may be transformed from a global coordinate system to a wellbore coordinate system. FIGS. 5A and 5B illustrate a schematic side view of the wellbore 102 that includes at least one perforation tunnel 110 with overlaid coordinate systems. In the global coordinate system 186, as shown in FIG. 5B, the x-axis aligns with north (true north), the y-axis aligns with east, and the z-axis is vertically downward (for example, into the Earth). This coordinate system 186 is denoted by global coordinate system (x_{G}, y_{G}, z_{G}). In some aspects, it is assumed that the azimuth of maximum principal stress is θ_{σH}, which is the angle turning clockwise from north to the maximum principle stress. Therefore, a rotation matrix is given by:

$${R}_{{σ}_{H} \to {x}_{G}} = ( \begin{matrix}cos {θ}_{{σ}_{H}} & - sin {θ}_{{σ}_{H}} & 0 \\ sin {θ}_{{σ}_{H}} & cos {θ}_{{σ}_{H}} & 0 \\ 0 & 0 & 1\end{matrix} ) . \qquad ( 14 )$$

In Eq. 14, θ_{σH} should be positive if the maximum principal stress, σ_{Hmax}, is clockwise to the axis, x_{G}, and is negative if it is counter clockwise to the axis, x_{G}. The equivalent stress tensor in the global coordinate system 186 that is rotated by a maximum stress angle θ_{σH} around the z-axis can be calculated by:

$${σ}^{G} = {R}_{{σ}_{H} \to {x}_{G}} {σ}_{Pr} {R}_{{σ}_{H} \to {x}_{G}}^{T} , \\ and \qquad ( 15 )$$

$$\{ \begin{matrix}{σ}_{xx}^{G} = {C}_{G}^{2} {σ}_{Hmax} + {S}_{G}^{2} {σ}_{Hmin} \\ {σ}_{yy}^{G} = {S}_{G}^{2} {σ}_{Hmax} + {C}_{G}^{2} {σ}_{Hmin} \\ {σ}_{zz}^{G} = {σ}_{V} \\ {τ}_{xy}^{G} = {C}_{G} {S}_{G} ( {σ}_{Hmax} - {σ}_{Hmin} ) \\ {τ}_{yz}^{G} = 0 \\ {τ}_{zx}^{G} = 0\end{matrix} , \qquad ( 16 )$$

where C_{G}=cos θ_{σH}, S_{G}=sin θ_{σH}. In some aspects, this is the same as the stress rotation used in the objective stress update procedures for large deformation analysis in computational mechanics. For a well survey, any point in the well trajectory can be determined by three parameters: measured depth, wellbore deviation angle α_{D}, and wellbore azimuth angle α_{A}.

A wellbore coordinate system 188 at any point along the well trajectory can be tracked and obtained by the following rotations about the global coordinate system x_{G}, y_{G}, z_{G}. For example, such rotations can include: (1) rotation of deviation angle α_{D} about the y_{G}-axis; and (2) rotation of azimuth angle α_{A} about z_{G}-axis. Then, the rotation matrix is given by:

$${R}_{G \to B} ( {α}_{A} , {α}_{D} ) = {R}_{y} ( {α}_{D} ) {R}_{z} ( {α}_{A} ) = ( \begin{matrix}cos {α}_{D} & 0 & - sin {α}_{D} \\ 0 & 1 & 0 \\ sin {α}_{D} & 0 & cos {α}_{D}\end{matrix} ) ( \begin{matrix}cos {α}_{A} & sin {α}_{A} & 0 \\ - sin {α}_{A} & cos {α}_{A} & 0 \\ 0 & 0 & 1\end{matrix} ) . \qquad ( 17 )$$

$$Eq . 17 may lead to :$$

$${R}_{G \to B} ( {α}_{A} , {α}_{D} ) = ( \begin{matrix}cos {α}_{D} cos {α}_{A} & cos {α}_{D} sin {α}_{A} & - sin {α}_{D} \\ - sin {α}_{A} & cos {α}_{A} & 0 \\ sin {α}_{D} cos {α}_{A} & sin {α}_{D} sin {α}_{A} & cos {α}_{D}\end{matrix} ) . \qquad ( 18 )$$

Then, the in-situ stress tensor along the wellbore trajectory (in other words, the trajectory of wellbore 102) can be projected onto the wellbore coordinate system 188 by:

$${σ}^{B , I} = {R}_{G \to B} ( {α}_{D} , {α}_{A} ) {σ}^{G} {R}_{G \to B}^{T} ( {α}_{D} , {α}_{A} ) = ( \begin{matrix}{σ}_{xx}^{B , I} & {τ}_{xy}^{B , I} & {τ}_{xz}^{B , I} \\ {τ}_{yz}^{B , I} & {σ}_{yy}^{B , I} & {τ}_{yz}^{B , I} \\ {τ}_{zx}^{B , I} & {τ}_{zy}^{B , I} & {σ}_{zz}^{B , I}\end{matrix} ) . \qquad ( 19 )$$

In Eq. 19, the superscript/represents the far field in-situ stress. Due to the lengthy expression of the stress components, the exact analytical expressions of Eq. 19 are not provided here but can be directly calculated through computer implementation.

The rotation matrix about the y-axis of the wellbore coordinate system 188 is given by:

$$R ( {α}_{y , B} ) = ( \begin{matrix}cos {α}_{y , B} & 0 & - sin {α}_{y , B} \\ 0 & 1 & 0 \\ sin {α}_{y , B} & 0 & cos {α}_{y , B}\end{matrix} ) . \qquad ( 20 )$$

Thus, the in-situ stresses are transformed from a global coordinate system to a wellbore coordinate system.

In some implementations, the computing system 120 transforms the in-situ stresses from the wellbore coordinate system 188 to a perforation coordinate system 190 through at least one rotation matrix. For example, in some aspects, rotating the y axis of the wellbore coordinate system 188 at any point along the wellbore axis by α_{y,B}=π/2, generates a particular perforation coordinate system 190. The corresponding rotation matrix is:

$${R}_{B \to P} ( {α}_{y , B} = \frac{π}{2} ) = ( \begin{matrix}0 & 0 & - 1 \\ 0 & 1 & 0 \\ 1 & 0 & 0\end{matrix} ) = {R}_{B \to P}^{1} . \qquad ( 21 )$$

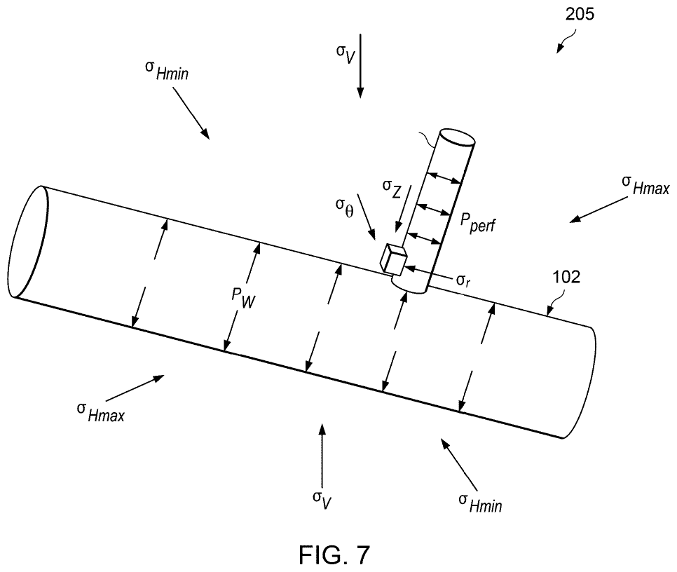

For example, FIG. 6 illustrates a schematic isometric view cross-section 200 of wellbore 102 that includes perforation tunnels 110 with an overlaid perforation coordinate system 190 at each perforation tunnel 110. As shown in cross-section 200, the perforation coordinate system 190 for different phase angles of perforation tunnels 110 can be rotated, for example, by: (1) rotating perforation phase angle α_{y,B}=π/2 about the y_{B}-axis of the wellbore coordinate system 188; and (2) rotation of phase angle α_{Z,B} about the z_{B}-axis of the wellbore coordinate system 188. In some aspects, for clustered perforations with a known perforation phase angle (for example, both phase angles about the y_{B}-axis and the z_{B}-axis are known), the rotation matrix from the wellbore coordinate system 188 (x_{B}, y_{B}, z_{B}) to the perforation coordinate system 190 (x_{P}y_{P}z_{P}) can be sequentially obtained by:

$${R}_{B \to P} ( {α}_{z , B} , {α}_{y , B} ) = {R}_{B \to P}^{1} {R}_{z , B} ( {α}_{z , B} ) = ( \begin{matrix}0 & 0 & - 1 \\ sin {α}_{Z , B} & cos {α}_{Z , B} & 0 \\ cos {α}_{Z , B} & sin {α}_{Z , B} & 0\end{matrix} ) . \qquad ( 22 )$$

In Eq. 22, α_{z,B} is the perforation phase angle rotating about the wellbore axis-z, which ranges from 0° to 360° and starts from the highest point of a wellbore cross section. Thus, the in-situ stresses are transformed from the wellbore coordinate system to a perforation coordinate system.

In some implementations, the computing system 120 determines one or more stresses (for example, a stress state) on a wellbore-perforation interface from the in-situ stresses (for example, and other contributed components) in the perforation coordinate system. For example, for wellbore 102, bottom hole pressure may act on the perforation tunnels 110 through two ways: (1) downhole pressure may be partially and radially transferred to the subsurface formation 104 through the casing 182-cement 184 layer(s) and eventually pressurize the subsurface formation 104, which induces stresses around the borehole-perforation interface; and (2) injecting fluid flows into the perforation tunnel 110 from the casing 182 and pressurizing the perforation wall directly.

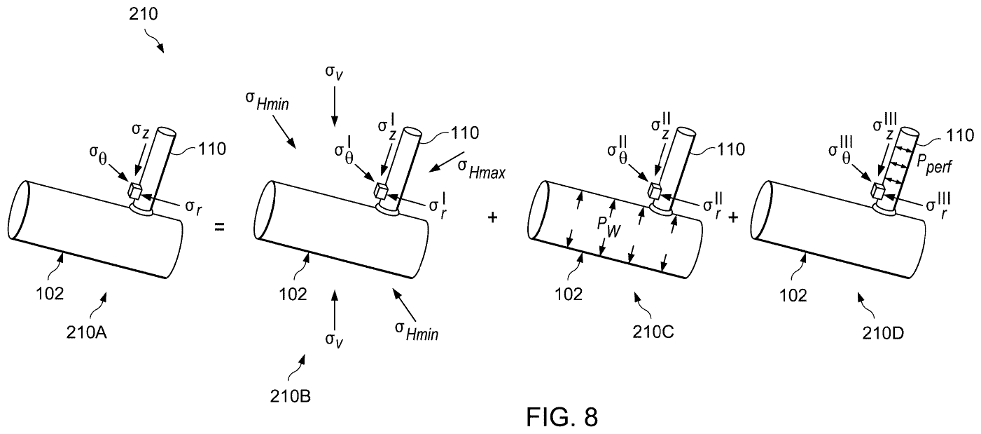

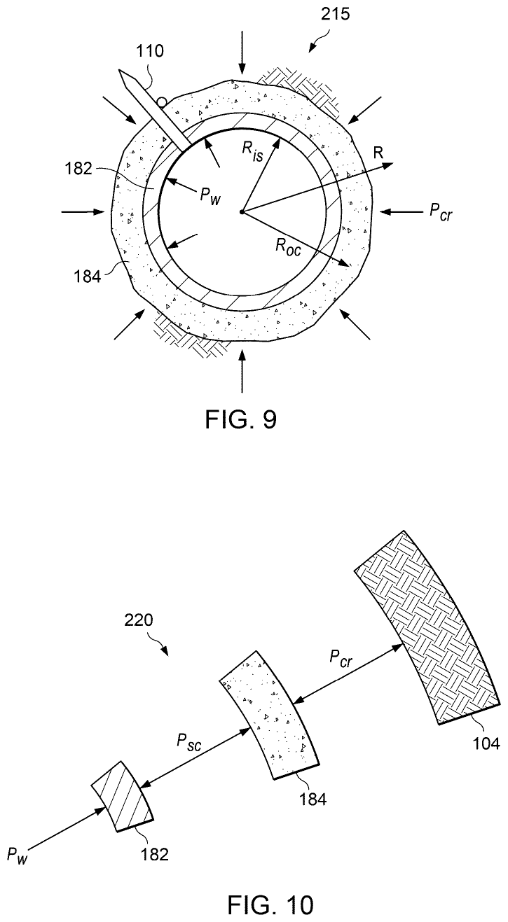

In some aspects, the deformation is limited to linear elasticity. Thus, the total stresses can be summed up (for example, superimposed) once the corresponding stresses induced by each load are calculated. For example, FIG. 7 illustrates a schematic isometric view 205 of a portion of the wellbore 102 with a particular perforation tunnel 110 and particular stresses and acting pressures. More specifically, view 205 shows the pressure loads that may be included to estimate the induced stresses around the wellbore-perforation interface. The induced stresses are also shown in FIG. 8, which illustrates an equation illustrated by a series 210 of schematic isometric views (210A, 210B, 210C, 210D) of a portion of wellbore 102 with a particular perforation tunnel 110 and particular stresses and pressures used to calculate a total effective stress through stress superposition. First, the stresses around the perforation tunnel 110 are induced by the far-field in-situ stress tensor (210B). Second, the borehole bottom hole pressure P_{w} can be partially and radially transferred to the rock of the subsurface formation 104 through the casing 182 and cement 184, which generates additional stresses over the wellbore-perforation interface (210C). Third, the pressure P_{perf} inside the perforation tunnel 110 induces stresses around the perforation tunnel 110, which may be a driving force to counter the combining stresses by the first and second parts and initiate a longitudinal fracture along the perforation tunnel 110 (210D).

In some aspects, the fluid pressure inside the perforation tunnel 110 might be different from the bottom hole pressure P_{w} inside the casing 182 if the perforation friction is not negligible. The resulting induced stresses by these loads are denoted by superscript as σ^{I}, σ^{II}, and σ^{III}, respectively, which are shown in FIG. 8.

From a mechanics point of view, the breakdown pressure of a perforation is a three dimensional mechanics problem and may be difficult to obtain a closed form analytical solution. Thus, in some aspects, the computing system 120 may use a plane strain approximation (satisfying the Kirsch equation) of the perforation tunnel 110. This approximation is feasible, because a ratio of perforation length to perforation diameter (L/D) is generally large in a real world example.

The breakdown pressure may be estimated based on the stress state at the perforation base, which is at the wellbore-perforation interface (for example, where the perforation 110 meets the wellbore 102) as marked in FIG. 8. The breakdown pressure refers to the bottom hole pressure P_{w}, whenever the hoop stress around the perforation base exceeds the tensile strength T of the rock of the subsurface formation 104. Based on the principle of stress superposition as shown in FIG. 8, the total effective stresses (210A) with respect to the perforation coordinate system are induced by the three parts as follows:

$${σ}^{P} = {σ}^{P , I} + {σ}^{P , II} + {σ}^{P , III} . \qquad ( 23 )$$

In Eq. 23, the superscript P refers to the perforation coordinate system 190 and superscripts I, II, III refer to the three loading cases (210B, 210C, 210D). The far field in-situ stress tensor σ^{P,I} around the perforation base in the perforation coordinate system 190 (x_{P}, y_{P}, z_{P}) is obtained as follows:

$${σ}^{P , I} = {R}_{B \to P} ( {α}_{z , B} , {α}_{y , B} ) {σ}^{B , I} {R}_{B \to P}^{T} ( {α}_{z , B} , {α}_{y , B} ) = ( \begin{matrix}{σ}_{xx}^{P , I} & {τ}_{xy}^{P , I} & {τ}_{xz}^{P , I} \\ {τ}_{yz}^{P , I} & {σ}_{yy}^{P , I} & {τ}_{yz}^{P , I} \\ {τ}_{zx}^{P , I} & {τ}_{zy}^{P , I} & {σ}_{zz}^{P , I}\end{matrix} ) . \qquad ( 24 )$$

Due to the lengthy expressions for the stress components, the exact analytical expressions are not provided here either and will be directly calculated in computer implementation. Based on Eq. 22, the far-field in-situ stresses in the perforation coordinate system 190 can be expressed in terms of stress tensor σ^{B} as follows

$$\{ \begin{matrix}{σ}_{xx}^{P , I} = {σ}_{zz}^{B , I} \\ {σ}_{yy}^{P , I} = {S}_{{α}_{z , B}}^{2} {σ}_{xx}^{B , I} + {C}_{{α}_{z , B}}^{2} {σ}_{yy}^{B , I} + 2 {C}_{{α}_{z , B}} {S}_{{α}_{z , B}} {τ}_{xy}^{B , I} \\ {σ}_{zz}^{P , I} = {C}_{{α}_{z , B}}^{2} {σ}_{xx}^{B , I} + {S}_{{α}_{z , B}}^{2} {σ}_{yy}^{B , I} + 2 {C}_{{α}_{z , B}} {S}_{{α}_{z , B}} {τ}_{xy}^{B , I} \\ {τ}_{xy}^{P , I} = - {S}_{{α}_{z , B}} {τ}_{xz}^{B , I} - {C}_{{α}_{z , B}} {τ}_{yz}^{B , I} \\ {τ}_{yz}^{P , I} = {S}_{{α}_{z , B}} {C}_{{α}_{z , B}} ( {σ}_{xx}^{B , I} + {σ}_{yy}^{B , I} ) + {τ}_{xy}^{B , I} \\ {τ}_{zx}^{P , I} = - {C}_{{α}_{z , B}} {τ}_{xz}^{B , I} - {S}_{{α}_{z , B}} {τ}_{yz}^{B , I}\end{matrix} . \\ In Eq . 25 , \\ {C}_{{α}_{z , B}} = cos {α}_{z , B} , {S}_{{α}_{z , B}} = sin {α}_{z , B} . \qquad ( 25 )$$

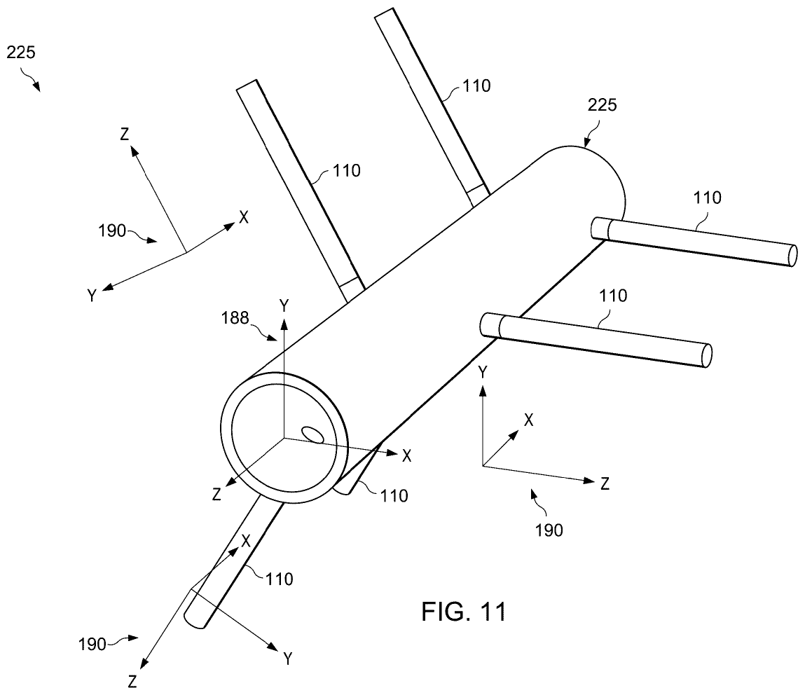

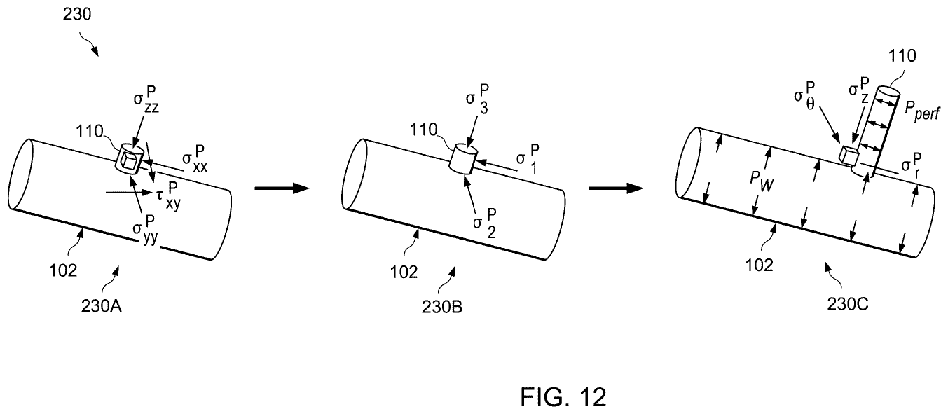

In some aspects, the computing system 120 accounts for impacts of the casing 182 and the cement 184 on the formation breakdown pressure. For example, as opposed to an open hole completion in which a downhole pressure will directly act on the rock of the subsurface formation, for a cased wellbore such as the wellbore 102, the bottom hole pressure P_{w} inside the casing 182 can be partially and radially transferred to the rock of the subsurface formation 104 through the casing 182 and the cement 184, which generate additional stresses over the wellbore-perforation interface. For example, FIGS. 9-10 illustrate these additional stresses.

FIG. 9 illustrates a schematic top view cross-section 215 of the wellbore 102 with casing 182 and cement 184 and the additional stresses, which are dependent on the magnitude of P_{cr} and related to the casing-cement-formation interaction. FIG. 10 illustrates an exploded schematic view 220 of a radial section of wellbore 102 with casing 182 and cement 184 and the additional pressure.

As shown in these figures, Rᵢₛ represents the casing inside radius, R_{oc} represents the cement outer radius, wellbore radius R_{w}=R_{oc}, fluid pressure P_{w} is inside the casing 182, casing-cement interface pressure is P_{sc}, and P_{cr} is pressure between the cement 184 and the rock of subsurface formation 104. In some aspects, the computing system 120 assumes that the casing-cement-rock are fully bonded without slip.

The casing-cement-rock interaction results in the final interface pressure acting on the subsurface formation 104, which should be less than inside casing pressure, P_{w}. In order to estimate this part induced stresses, the computing system 120 uses an analytical elastic solution for calculating the cement-formation interface pressure, P_{cr}. For example, the internal pressure loading of the casing 182 can be considered as an axisymmetric plane strain problem even for a deviated well trajectory such as wellbore 102. After removing the in-situ stresses effect, the analytical solution for the cement-formation interface pressure P_{cr} can be obtained as follows:

$${P}_{cr} = \frac{- {C}_{21} {b}_{1} {P}_{W}}{{C}_{11} {C}_{22} - {C}_{21} {C}_{12}} = {β}_{cr} {P}_{w} . \qquad ( 26 )$$

$$In Eq . 26 :$$

$${C}_{11} = \frac{( 1 + {μ}_{s} ) [ ( 1 - 2 {μ}_{s} ) {R}_{os}^{2} + {R}_{is}^{2} ] {R}_{os}}{{R}_{os}^{2} - {R}_{is}^{2}} + \frac{{E}_{s} ( 1 + {μ}_{s} ) [ ( 1 - 2 {μ}_{c} ) {R}_{ic}^{2} + {R}_{oc}^{2} ] {R}_{ic}}{{E}_{c} ( {R}_{oc}^{2} - {R}_{ic}^{2} )} , \qquad ( 27 )$$

$${C}_{12} = - \frac{2 {E}_{s} ( 1 - {μ}_{c}^{2} ) {R}_{oc}^{2} {R}_{ic}}{{E}_{c} ( {R}_{oc}^{2} - {R}_{ic}^{2} )} , \qquad ( 28 )$$

$${C}_{21} = - \frac{2 {E}_{f} ( 1 - {μ}_{c}^{2} ) {R}_{ic}^{2} {R}_{oc}}{{E}_{c} ( {R}_{oc}^{2} - {R}_{ic}^{2} )} , \qquad ( 29 )$$

$${C}_{22} = \frac{{E}_{f} ( 1 + {μ}_{c} ) [ ( 1 - 2 {μ}_{c} ) {R}_{oc}^{2} + {R}_{ic}^{2} ] {R}_{oc}}{{E}_{c} ( {R}_{oc}^{2} - {R}_{ic}^{2} )} + ( 1 + {μ}_{f} ) {R}_{w} , \qquad ( 30 )$$

$${b}_{1} = \frac{2 ( 1 - {μ}_{s}^{2} ) {R}_{is}^{2} {R}_{os}}{( {R}_{oc}^{2} - {R}_{ic}^{2} )} , \\ and \qquad ( 31 )$$

$${β}_{cr} = \frac{- {C}_{21} {b}_{1}}{{C}_{11} {C}_{22} - {C}_{21} {C}_{12}} . \qquad ( 32 )$$

In the above equations, subscript “s” refers to the casing, “f” refers to the formation or rock, and “c” refers to the cement. After obtaining the magnitude of pressure acting on the rock transferred through the casing 182 and cement 184, it can be approximately assumed that an axisymmetric plane strain condition exists along the wellbore axis direction under the load of interface pressure. Thus, the stress distribution near the wellbore 102 can be given as follows:

$$\{ \begin{matrix}{σ}_{R}^{B , II} = {P}_{cr} \frac{{R}_{oc}^{2}}{{R}^{2}} \\ {σ}_{θ}^{B , II} = - {P}_{cr} \frac{{R}_{oc}^{2}}{{R}^{2}}\end{matrix} \qquad ( 33 )$$

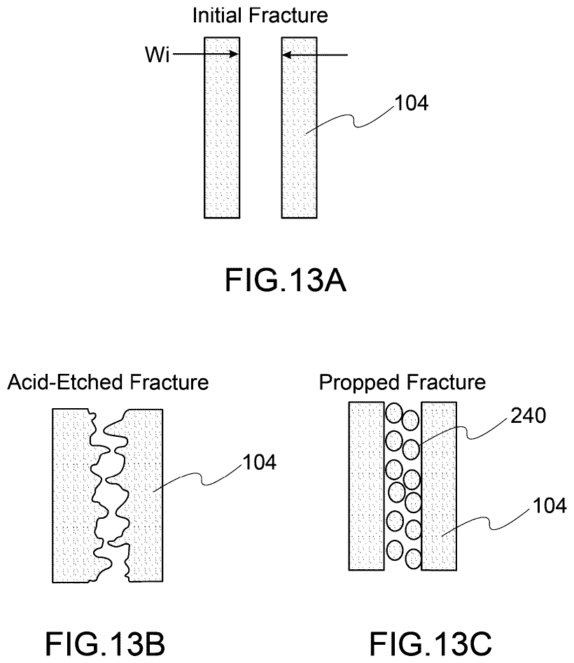

The above radial and circumferential stresses are constant regardless of angle θ about the axis of wellbore 102. This is convenient for applying stress superposition to calculate the total breakdown pressure when projecting them onto the perforation coordinate systems 190 (for example, as shown in FIGS. 6, 9, and 10). In some aspects, the stress components of σ_{R}^{B,II}, σ_{θ}^{B,II} and σ_{Z}^{B,II} are calculated with respect to the wellbore 102, which has a different orientation (and coordinate system 188) from the perforation coordinate system 190 (x_{P}, y_{P}, z_{P}) as shown in FIG. 11 (and FIG. 5A). For example, FIG. 11 illustrates an isometric view 225 of the wellbore 102 (for example, horizontal portion 102B) with perforation tunnels 110 and overlaid coordinate systems for the wellbore 102 and perforation tunnels 110.

As shown in FIG. 11 (and FIG. 5A), in the perforation coordinate system 190 (x_{P}, y_{P}, z_{P}), σ_{θ}^{B,II} aligns in the same direction of the y_{P}-axis, σ_{Z}^{B,II} aligns in the direction of the x_{P}-axis, and σ_{R}^{B,II} aligns in the direction of z_{P}-axis. Based on the orientations of the coordinate systems attached to wellbore and perforations as shown in these figures, it leads to the following relationships:

$${σ}_{xx}^{P , II} = {σ}_{zz}^{B , II} = 0 , \qquad ( 34 )$$

$${σ}_{yy}^{P , II} = {σ}_{θ θ}^{B , II} = - {P}_{cr} \frac{{R}_{oc}^{2}}{{R}^{2}} = - {P}_{cr} , \\ and \qquad ( 35 )$$

$${σ}_{zz}^{P , II} = {σ}_{rr}^{B , II} = {P}_{cr} \frac{{R}_{oc}^{2}}{{R}^{2}} = {P}_{cr} . \qquad ( 36 )$$

Calculation of σ^{III} stress tensor around the perforation tunnel 110 (for example at the perforation interface) may be related to the fluid pressure inside the perforation tunnel 110. For each perforation tunnel 110, it can be reasonably considered as an axisymmetric loading case and with a plane strain condition along the perforation axis, zₚ. Therefore, the expression is the same to Eq. 33, but the radial direction should be in the perforation radial direction (and it is also differentiated by lower case r):

$${σ}_{r}^{P , III} = {P}_{perf} \frac{{r}_{p}^{2}}{{r}^{2}} and {σ}_{θ}^{P , III} = - {P}_{perf} \frac{{r}_{p}^{2}}{{r}^{2}} . \qquad ( 37 )$$

To calculate the fluid pressure acting on the perforation tunnel 110, the pressure loss across the perforation entry is accounted for, which may be important for hydraulic fracturing pump schedule design. For example, a sharp-edge orifice equation may be used to estimate the pressure drop as follows:

$$Δ {P}_{perf} = \frac{0.2369 ρ}{{d}^{4} {C}_{d}^{2}} {( \frac{Q}{N} )}^{2} , \qquad ( 38 )$$

where ρ is fluid density in lb/gal; d is the initial perforation diameter in inches; C_{d} is the perforation coefficient of discharge; Q is the flow rate in bbl/min; and N is the number of perforation tunnels 110.

The total fluid pressure inside the perforation tunnel may be expressed as:

$${P}_{perf} = {P}_{w} - Δ {P}_{perf} = {β}_{perf} {P}_{w} , \\ and \qquad ( 39 )$$

$${β}_{perf} = 1 - \frac{0.2369 ρ}{{d}^{4} {C}_{d}^{2}} {( \frac{Q}{N} )}^{2} . \qquad ( 40 )$$

In Eq. 39, β_{perf}∈[0, 1] represents a perforation fraction of wellbore pressure effectively transferred to a perforation hole and that acts on the perforation wall to counter the induced hoop stresses by in-situ stresses and P_{cr}, and initiate the hydraulic fracture. Before rock breakdown and fracture initiation, P_{perf} can be assumed to be equal to wellbore pressure, P_{w}, if friction loss is negligible. Otherwise, the internal pressure acting on a perforation wall may be adjusted based on the perforation quality. For example, perforation quality (for example, accounted for by β_{perf}) can significantly impact the breakdown issue, which may be accounted for calculating breakdown pressure.

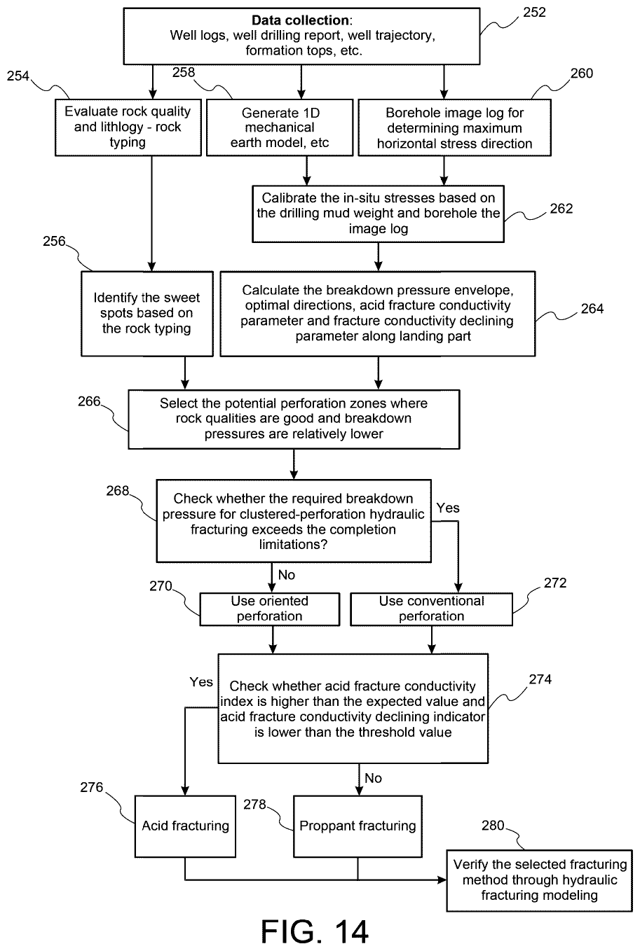

In some implementations, the computing system 120 calculates one or more hoop stresses at a perforation tunnel wall of the perforation tunnel from the determined stresses on the wellbore-perforation interface. For example, in some aspects, the determined stresses are used to calculate the hoop stresses at the wellbore-perforation interface. For instance, FIG. 12 illustrates a series 230 of schematic isometric views (230A, 230B, and 230C) of a portion of the wellbore with a particular perforation tunnel 110 and particular stresses used to calculate a hoop stress around the perforation tunnel 110. View 230A shows a first sub-step that includes adding the induced stresses of loading cases I and II (210B and 210C) together. Limiting R=R_{oc}=R_{w} results in the stresses within the perforation-wellbore interface as follows:

$$\{ \begin{matrix}{σ}_{xx}^{P , I - II} = {σ}_{xx}^{P , I} + {σ}_{xx}^{P , II} = {σ}_{zz}^{B , I} + {σ}_{zz}^{B , II} = {σ}_{zz}^{B , I} \\ {σ}_{yy}^{P , I - II} = {σ}_{yy}^{P , I} + {σ}_{yy}^{P , II} = {S}_{{α}_{z , B}}^{2} {σ}_{xx}^{B , I} + {C}_{{α}_{z , B}}^{2} {σ}_{yy}^{B , I} + \\ 2 {C}_{{α}_{z , B}} {S}_{{α}_{z , B}} {τ}_{xy}^{B , I} - {P}_{cr} \\ {σ}_{zz}^{P , I - II} = {σ}_{zz}^{P , I} + {σ}_{rr}^{P , II} = {C}_{{α}_{z , B}}^{2} {σ}_{xx}^{B , I} + {S}_{{α}_{z , B}}^{2} {σ}_{yy}^{B , I} + \\ 2 {C}_{{α}_{z , B}} {S}_{{α}_{z , B}} {τ}_{xy}^{B , I} + {P}_{cr} \\ {τ}_{xy}^{PI - II} = - {S}_{{α}_{z , B}} {τ}_{xz}^{B , I} - {C}_{{α}_{z , B}} {τ}_{yz}^{B , I} \\ {τ}_{yz}^{P , I - II} = {S}_{{α}_{z , B}} {C}_{{α}_{z , B}} ( {σ}_{xx}^{B , I} + {σ}_{yy}^{B , I} ) + {τ}_{xy}^{B , I} \\ {τ}_{zx}^{P , I - II} = - {C}_{{α}_{z , B}} {τ}_{xz}^{B , I} - {S}_{{α}_{z , B}} {τ}_{yz}^{B , I}\end{matrix} \qquad ( 41 )$$

For the purpose of mathematical symbolic simplification and computer implementation, Eq. 41 can be further simplified as:

$$\{ \begin{matrix}{σ}_{xx}^{P , I - II} = {S}_{xx}^{0} \\ {σ}_{yy}^{P , I - II} = {S}_{yy}^{0} - {P}_{cr} = {S}_{yy}^{0} - {β}_{cr} {P}_{w} \\ {σ}_{zz}^{P , I - II} = {S}_{zz}^{0} {P}_{cr} = {S}_{zz}^{0} + {β}_{cr} {P}_{w} \\ {τ}_{xy}^{P , I - II} = {T}_{xy}^{0} \\ {τ}_{yz}^{P , I - II} = {T}_{yz}^{0} \\ {τ}_{zx}^{P , I - II} = {T}_{zx}^{0}\end{matrix} \qquad ( 42 )$$

$$\{ \begin{matrix}{S}_{xx}^{0} = {σ}_{zz}^{B , I} \\ {S}_{yy}^{0} = {S}_{{α}_{z , B}}^{2} {σ}_{xx}^{B , I} + {C}_{{α}_{z , B}}^{2} {σ}_{yy}^{B , I} + 2 {C}_{{α}_{z , B}} {S}_{{α}_{z , B}} {τ}_{xy}^{B , I} \\ {S}_{zz}^{0} = {C}_{{α}_{z , B}}^{2} {σ}_{xx}^{B , I} + {S}_{{α}_{z , B}}^{2} {σ}_{yy}^{B , I} + 2 {C}_{{α}_{z , B}} {S}_{{α}_{z , B}} {τ}_{xy}^{B , I} \\ {T}_{xy}^{0} = - {S}_{{α}_{z , B}} {τ}_{xz}^{B , I} - {C}_{{α}_{z , B}} {τ}_{yz}^{B , I} \\ {T}_{yz}^{0} = {S}_{{α}_{z , B}} {C}_{{α}_{z , B}} ( {σ}_{xx}^{B , I} + {σ}_{yy}^{B , I} ) + {τ}_{xy}^{B , I} \\ {T}_{zx}^{0} = - {C}_{{α}_{z , B}} {τ}_{xz}^{B , I} - {S}_{{α}_{z , B}} {τ}_{yz}^{B , I}\end{matrix} \qquad ( 43 )$$

Eq. 43 gives the stresses acting on the wellbore-perforation interface (as shown in FIG. 8), which are induced by the far field in-situ stresses and interface pressure P_{cr}.

View 230B shows a second sub-step that includes calculating the local maximum and minimum stresses in the perforation base due to loading cases I and II. The local principal stresses in the perforation coordinate system plane, x_{P}y_{P}, on the wellbore-perforation interface that surrounds the perforation tunnel can be given by:

$$\{ \begin{matrix}{σ}_{m ax}^{P} = \frac{{σ}_{xx}^{P , I - II} + {σ}_{yy}^{P , I - II}}{2} + \sqrt{{( \frac{{σ}_{xx}^{P , I - II} - {σ}_{yy}^{P , I - II}}{2} )}^{2} + {( {τ}_{xy}^{P , I - II} )}^{2}} \\ {σ}_{m i n}^{P} = \frac{{σ}_{xx}^{P , I - II} + {σ}_{yy}^{P , I - II}}{2} - \sqrt{{( \frac{{σ}_{xx}^{P , I - II} - {σ}_{yy}^{P , I - II}}{2} )}^{2} + {( {τ}_{xy}^{P , I - II} )}^{2}}\end{matrix} , \\ or \qquad ( 44 )$$

$${σ}_{m ax}^{P} = \frac{{s}_{xx}^{0} + {s}_{yy}^{0} - {P}_{cr}}{2} + \sqrt{{( \frac{{s}_{xx}^{0} - {s}_{yy}^{0} + {P}_{cr}}{2} )}^{2} + {( {T}_{xy}^{0} )}^{2}} , \\ and \qquad ( 45 )$$

$${σ}_{m i n}^{P} = \frac{{s}_{xx}^{0} + {s}_{yy}^{0} - {P}_{cr}}{2} - \sqrt{{( \frac{{s}_{xx}^{0} - {s}_{yy}^{0} + {P}_{cr}}{2} )}^{2} + {( {T}_{xy}^{0} )}^{2}} . \qquad ( 46 )$$

View 230C shows a third sub-step that includes calculating the hoop stress around the perforation tunnel 110. Following the conditions satisfying the Eqs. 7 and 9, a plane strain condition along the perforation axis, z_{P}, can be assumed. Therefore the hoop stress with respect to the perforation tunnel 110 can be obtained as follows:

$${σ}_{θ} = ( {S}_{xx}^{0} + {S}_{yy}^{0} - {P}_{cr} ) - 2 cos 2 {θ}_{p} \sqrt{{( {S}_{xx}^{0} - {S}_{yy}^{0} + {P}_{cr} )}^{2} + 4 {T}_{xy}^{{0}^{2}}} - ( {P}_{perf} - {P}_{0} ) . \qquad ( 47 )$$

Eq. 47 adds the induced stress by P_{cr}. Since, in some aspects, the rock breakdown due to the fracturing fluid likely initiates at the perforation base along the direction of σₘₐₓ^{P} with θₚ=0, this leads to the hoop stress as follows:

$${σ}_{θ} = ( {S}_{xx}^{0} + {S}_{yy}^{0} - {P}_{cr} ) - 2 \sqrt{{( {S}_{xx}^{0} - {S}_{yy}^{0} + {P}_{cr} )}^{2} + 4 {( {T}_{xy}^{0} )}^{2}} - ( {P}_{perf} - {P}_{0} ) . \qquad ( 48 )$$

The hydraulic fracture generally initiates when the hoop stress around the perforation tunnel 110 exceeds the rock tensile strength, T. Based on the stress sign convention, it follows:

$$( {S}_{xx}^{0} + {S}_{yy}^{0} - {P}_{cr} ) - 2 \sqrt{{( {S}_{xx}^{0} - {S}_{yy}^{0} + {P}_{cr} )}^{2} + 4 {( {T}_{xy}^{0} )}^{2}} - ( {P}_{perf} - {P}_{0} ) = - T , \qquad ( 49 )$$

$$a = {S}_{xx}^{0} + {S}_{yy}^{0} , \\ and \qquad ( 50 )$$

$$b = {S}_{xx}^{0} - {S}_{yy}^{0} , \qquad ( 51 )$$

Eq. 49 can be simplified as:

$$a + T + {P}_{0} - ( {P}_{cr} + {P}_{perf} ) - 2 \sqrt{{( b + {P}_{cr} )}^{2} + 4 {( {T}_{xy}^{0} )}^{2}} = 0 , \qquad ( 52 )$$

with:

$$c = a + T + {P}_{0} , \qquad ( 53 )$$

$${P}_{perf} = {β}_{perf} {P}_{w} , \\ and \qquad ( 54 )$$

$${P}_{cr} = {β}_{cr} {P}_{w} . \qquad ( 55 )$$

In some implementations, the computing system 120 determines a breakdown pressure for the subsurface formation based at least in part on the calculated one or more hoop stresses. In some aspects, the computing system 120 solves a non-linear equation to determine the breakdown pressure. For example, based on the calculated hoop stresses, the breakdown pressure, P_{w}, of the subsurface formation 104 into which a cased, deviated wellbore (wellbore 102) is formed may be calculated according to:

$$( {β}_{perf}^{2} + 2 {β}_{cr} {β}_{perf} - 3 {β}_{cr}^{2} ) {P}_{w}^{2} - ( 2 c {β}_{cr} + 8 b {β}_{cr} + 2 c {β}_{perf} ) {P}_{w} + {c}^{2} - 4 {b}^{2} - 16 {( {T}_{xy}^{0} )}^{2} = 0. \qquad ( 56 )$$

The result of this calculation of P_{w} should be a positive number. As shown, Eq. 56 is a quadratic equation and the breakdown pressure may not be given by an explicit expression (as is the case for conventional, vertical, open hole wellbores), namely, because of the casing-cement interaction and estimating the interface pressure, Per, can be estimated by the computing system 120.

The computing system 120 receives one or more parameters as input and produces a scalar estimate of breakdown pressure (P_{w}) as output. The breakdown pressures are determined based at least in part on the in-situ stresses in the perforation coordinate system. The breakdown pressures are determined based at least in part on hoop stresses at a perforation tunnel wall of the at least one perforation tunnel.

In some examples, the parameters include aspects related to in-situ stresses of the subsurface formation 104 and orientations of the in-situ stresses (for example, the maximum horizontal in-situ stress (S_{Hmax}), the minimum horizontal in-situ stress (S_{Hmin}), the orientation angle of the maximum or minimum horizontal in-situ stress, the vertical stress (for example, the S_{V} (total stress) and/or the σ_{V} (effective stress), etc.).

In some examples, the parameters include mechanical properties (for example, static and/or dynamic Young's modulus of the casing 182, the cement 184, and/or the subsurface formation 104, the static and/or dynamic Poisson's ratio of the casing 182, the cement 184, and/or the subsurface formation 104, etc.), fluid properties of the fracking liquid (for example, the fluid density (φ, the perforation coefficient of discharge (C_{d}), the flow rate (Q), perforation fraction (β_{perf}), etc.) and mechanical failure criteria (for example, the tensile strength (T) of the subsurface formation 104).

In some examples, the parameters include geometric parameters (for example, the radius of the wellbore wall (R_{w}), the casing inside radius (Rᵢₛ), the casing outside radius (R_{oc}), the initial perforation diameter (d), the number of perforation tunnels (N), the measured depth within the wellbore, etc.) and geometric orientations of the perforation tunnels 110 (for example, the phase angle about the y_{B}-axis and/or the phase angle about the z_{B}-axis for each of the perforation tunnels), and/or geometric orientations of the wellbore 102 (for example, the deviation angle α_{D} about the y_{G}-axis and/or the azimuth angle α_{A} about z_{G}-axis, etc.).

In some examples, the computing system 120 determines the breakdown pressure for perforation clusters at different depths within the wellbore 102 (e.g., as a function of depth). For example, the computing system 120 determines breakdown pressures along the measured depth (or trajectory) of the wellbore 102 and also along perforation orientations. In some examples, the parameters and/or the stresses depend on the measured depth (or trajectory).

In some implementations, the computing system 120 determines a range of breakdown pressures for a particular formation layer (e.g., the lowest and highest breakdown pressures at each measured depth of the formation layer). Then, the computing system 120 defines a breakdown pressure envelope as a range of pressures between the lowest limit and highest limit for each measured depth. The computing system 120 determines the breakdown pressure as the lowest breakdown pressure at each measured depth, which requires the perforations to be shot at the determined optimal perforation directions.

### Perforation Locations

Identifying the best perforation locations in the well is also important. The computing system 120 determines optimal perforation locations based on the rock typing results and breakdown pressure envelopes of the formation. In some implementations, the computing system 120 performs this determination by (i) identifying depths with rock types above a threshold (e.g., above 4 or 5) which represent the areas where it is desirable to fracture and stimulate the formation, and (ii) identifying depths with either the lowest breakdown pressures or breakdown pressures below a threshold (e.g., below an average threshold pressure across all measured depths) to increase the chances that breakdown can be achieved by the completion and surface treating equipment (e.g., casing strength, pumping pressure, etc.). In some examples, the result of this determination is a plurality of depth ranges of the wellbore that are ideal for perforations. Determining the optimal perforation locations can also be based on other parameters of the formation, as described below.

### Perforation Strategy

After identifying the desired perforation locations, the computing system 120 identifies the depths in which the reservoir rock quality is relatively good and the breakdown pressure is relatively low and determines that conventional perforation should be applied at these depths. In some examples, the computing system 120 determines that the perforation cluster should use the same perforation diameter for each perforation tunnel and the phase angle should increase spirally at an increment of 60° for the depths in which the reservoir rock quality is relatively good and the breakdown pressure is relatively low.

However, in some situations, the required breakdown pressure might exceed the bottom hole pressure limitations that the completion can safely provide. In such situations, the computing system 120 determines that an oriented perforation should be used. The optimal perforation directions (perforation azimuth, perforation phase angles) can be calculated by the computing system 120 where the breakdown pressure is the lowest compared to other perforation directions at a measured depth.

Based on solid mechanics, oriented perforations can initiate hydraulic fractures with the lowest breakdown pressure at a given measured depth. This method can further increase the hydraulic fracturing success rate, which is beneficial for hydraulic fracturing treatments. Depending on the well trajectory, oriented perforation strategy may vary along the depth of the wellbore.

### Fracturing Treatments

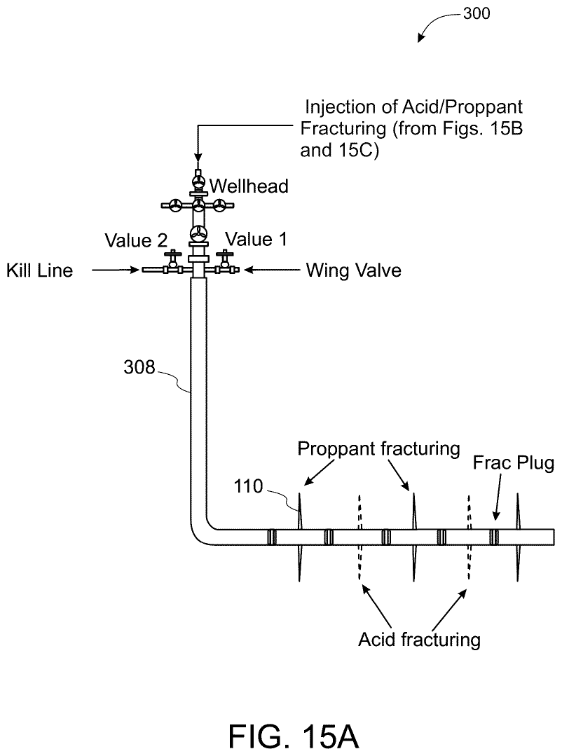

FIG. 13A is a diagram of an initial fracture in the formation 104 that has been formed by pumping a viscous pad fluid. The fracture has a fracture width, wᵢ. After the initial fracture has been formed, a fracturing fluid is typically pumped into the wellbore 102 to stimulate the formation, avoid or reduce the chances of the fractures closing, and/or widen or create additional flow paths in the formation 104. Fracturing methods include acid fracturing or proppant fracturing.

FIG. 13B is a diagram of the fracture of FIG. 13A after it has been subjected to acid fracturing. In some examples, an acid (e.g., plain acid, gelled acid, cross-linked acid, or emulsified acid) is pumped into the wellbore 102 to create a conductive acid fracture in the formation 104. In some cases, various acidic/non-acidic (e.g., viscosified gel) fluids are injected into the formation 104 at the bottom hole pressure above the fracture breakdown pressure to initiate hydraulic fractures. In some examples, hydrochloric acid (HCl) mixed with additives is used for acid fracturing of carbonate formations.

During acid injection, several processes take place simultaneously. First, acid flows along the fracture drive fracture propagating deep into the formation 104. Second, acid reacts with the rock at the fracture surfaces, creating surface irregularities or morphologies on the fracture faces. During this process, part of the rock at the fracture surfaces is dissolved due to chemical reaction and etching of acid, which creates channels and leaves behind undissolved portions of the rock possibly acting as pillars. Third, acid penetrates into the formation 104 in the direction perpendicular to the fracture, forming wormholes and leading to leaking off into the formation 104. The stimulated well performance after acid fracturing treatment is dictated by the final fracture conductivity when the injection is stopped and formation closure pressures are applied to the created fractures. In acid fracturing, conductivity is achieved with etched fracture faces since no proppant is used as in proppant fracturing.

Parameters affecting the final fracture conductivity include the amount of rock dissolved, the pattern of rock dissolution, and rock strength. Many factors impact the amount of rock dissolved and the etching pattern created during acid fracturing, such as acid type and concentration, acid contact time with the formation, reaction temperature, and injection rate. In addition to these reaction kinetic parameters, fracture conductivity is also highly affected by rock permeability and porosity and mineral composition, rock strength, and formation closure stress. In some cases, the stimulation efficiency of acid fracturing in deep and tight carbonate reservoirs may not be ideal due to quickly declining fracture conductivity.

FIG. 13C is a diagram of a fracture that has been subjected to proppant fracturing. A proppant 240 is pumped into the wellbore 102. The proppant 240 is a solid material, typically sand, treated sand, or man-made ceramic materials.

In some situations, especially mineralogy dominated cases, proppant fracturing treatments can be a better solution than acid fracturing treatments. For carbonate reservoirs, the performance of acid fracturing is compared with proppant fracturing in terms of long term fracture conductivity before determining which stimulation method should be used. In some examples, the computing system 120 determines whether proppant fracturing is an appropriate alternative stimulation technique to acid fracturing. Determination of which treatment type to be utilized is not straight forward and requires consideration of several factors implemented by the computing system 120. The systems and methods described in this disclosure determine the optimal treatment type for a particular depth interval of the formation 104 based on an acid fracture conductivity parameter (or indicator) (AFCI) and a fracture conductivity declining parameter (or indicator) (FCDI) that can vary as a function of a wellbore trajectory of a well. In some examples, the acid fracture conductivity parameter and the fracture conductivity declining parameter (or indicator) are referred to as being “depth-specific” or “depth-dependent” since they can vary as a function of the wellbore trajectory of the well.

### Acid Fracture Conductivity Parameter (AFCI)

Carbonate formations generally consist of two major components: calcite and dolomite. Some experimental studies indicate that there is a difference between the behavior of limestone and dolomite rocks when stimulated with HCl acids. Dolomite is denser and also has a more compressed packing system than calcite, suggesting that the dolomite is more compact. This explains the higher elastic and sound velocity properties of dolomite. Also, the compressive strength of dolomite is higher than calcite. The solubility of dolomite in water and acid is lower than that of calcite. For this reason, it is important to differentiate the stimulation strategy at the landing depth of the reservoir based on the volume fraction of minerals. For deep and tight carbonate reservoirs, the computing system 120 evaluates the acid fracture conductivity, which is based on the formation embedment strength and formation closure stress.

In some examples, the computing system 120 determines fracture conductivity of the formation based on a fracture closure stress, rock embedment stress, and dissolved rock equivalent conductivity. In some examples, the computing system 120 determines fracture conductivity (wk_{f}) by evaluating the following equation:

$${wk}_{f} = {C}_{1} {e}^{- {C}_{2} {σ}_{c}} , \\ where : \\ \begin{matrix}{C}_{1} = 0.26 {5 [ DREC ]}^{0.822} , DREC = 3.73 \times 1 {0}^{8} {w}_{i}^{3} ; \\ {C}_{2} = ( 13.9 - 1.3 ln {S}_{em} ) \times 1 {0}^{- 3} for {S}_{em} < 20000 psi ; \\ {C}_{2} = ( 3.8 - 0 \text{.28} ln {S}_{em} ) \times 1 {0}^{- 3} for {S}_{em} > 20000 psi ;\end{matrix} \qquad ( 57 )$$

wk_{f} is the fracture conductivity in millidarcy inch (md-in); σ_{c} is the fracture closure pressure in pounds per square inch (psi); DREC is the dissolved rock equivalent conductivity in md-in; Sₑₘ is the rock embedment strength of the formation in psi; wᵢ is the fracture width due to acid etching; and ln(●) is the natural log operator.

Fracture closure pressure is the fluid pressure needed to initiate the opening of a fracture. This is contrast to breakdown pressure, which is the fluid pressure required to initiate a fracture from a perforation tunnel in a formation. In some examples, the fracture closure pressure is equal to the minimum in-situ stress computed from mechanical earth models (e.g., 1 dimensional (1D) mechanical earth models), and calibrated with borehole image logs, and drilling mud weight.

The rock embedment strength represents the force required to push a metal sphere a certain distance into the surface of a rock sample. In some examples, the rock embedment strength is related to and measured by unconfined compressive stress testing.

Fracture width refers to the opening width of a fracture along a normal direction. An example fracture width is shown in FIG. 13A. In some examples, fracture width is determined by numerical simulation. In some examples, fracture width is determined by lab experiments of a core plug of the formation.

In some implementations, the computing system 120 determines the acid fracture conductivity parameter (AFCI) by setting wᵢ equal to 0.2 in Equation (1) above and evaluating the following equation:

$$AFCI = 4.3063 \times 1 {0}^{4} {e}^{- {C}_{2} {σ}_{c}} , \qquad ( 58 )$$

In some examples, the computing system 120 determines the depth-specific acid fracture conductivity parameter based on the depth-specific data of the formation 104 from the logging device 112, a 1D mechanical Earth model representing the formation 104, and/or a laboratory test of a core plug representing the formation 104.

High AFCI values represent formations with good acid fracture conductivity properties where acid fracturing is preferred. Low AFCI formations represent formations where acid fracturing may not be ideal and instead proppant fracturing is preferred. In some examples, the computing system 120 determines that proppant fracturing is preferred when the depth-specific acid fracture conductivity parameter is below a first threshold (e.g., below 0.15 or 0.2), and that acid fracturing treatment is preferred when the depth-specific acid fracture conductivity parameter is above the first threshold.

### Fracture Conductivity Declining Parameter (FCDI)

The computing system 120 also determines a depth-fracture conductivity declining parameter (FCDI) of the formation 104. The fracture conductivity declining parameter represents a measure of the conductivity decline due to uneven etching fracture surfaces of the formation. In some examples, the computing system 120 determines the depth-specific fracture conductivity declining parameter by evaluating the following equation:

$$FCDI = \frac{{σ}_{c}}{UCS} , \qquad ( 59 )$$

where σ_{c} is the fracture closure pressure in psi, and UCS is the unconfined compressive strength of the formation 104 in psi.

In some examples, the computing system 120 determines the depth-specific fracture conductivity declining parameter based on the mechanical Earth model representing the formation 104, and a laboratory test of a core plug representing the formation 104.

Acid Fracturing versus Proppant Fracturing

The success of the acid fracturing depends on the created conductivity being sustained under formation closure pressure. The fracture surface asperity or morphology due to uneven etching will contribute to the final conductivity. However, acid fracture conductivity is the resulting competition between etching thickness of fracture surface and the weakening of the rock compressive strength after the acid fracturing.

Dolomite and calcite are the two main carbonate minerals. However, their reaction and corrosion rates with acids and their response to acid fracturing are different. A significant error on fracturing results can be encountered by assuming that they have the same resulting fracture conductivity characteristics. For carbonate reservoir, the dissolved rock equivalent conductivity generates the maximum fracture conductivity at the moment formation closure pressure reaching zero. After fluid leak-off or flow back, rock embedment strength controls the conductivity, which declines with increasing fracture closure pressure. To achieve a good acid fracturing treatment, a high dissolved rock equivalent conductivity (DREC) and a high rock embedment strength Sₑₘ that can be maintained for a long period of time are strongly desired. Unfortunately, high dissolved rock equivalent conductivity and high rock embedment strength for carbonate are difficult to obtain simultaneously. This appears to be the result of the weakening of the rock structure at the face of the fracture with increased acid exposure due to acid dissolving. The systems and methods of this disclosure consider these factors when selecting the fracturing method for a particular depth interval of the wellbore.

For limestone (calcite) formations, the final fracture conductivity is highly dependent on rock embedment strength, even though the dissolved rock volume and initial fracture conductivity are high. However, the rock embedment strength is relatively lower than that of dolomite for same acid fracturing condition. This can negatively impact the fracture conductivity for calcite dominated carbonate after acid fracturing. For this reason, it might be beneficial to use proppant fracturing. In case acid fracturing is used, the upper limit of HCl concentration is also preferably controlled.

For carbonate reservoirs with high volume fraction of dolomite, the final fracture conductivity is highly dependent on the dissolved rock volume. Dolomite has a high rock embedment strength but relatively poor dissolving rock performance so a higher concentration of HCl acid fluid can be used for acid fracturing. For this reason, the computing system 120 uses acid fracturing for depth intervals of the wellbore with high volume fraction of dolomite mineral and with relatively high concentrations acid fluid (e.g., 28% HCl). The impact on the final acid conductivity is evaluated using Eq. 57. The computing system 120 uses proppant fracturing for depth intervals with lower acid fracture conductivity parameter and high fracture conductivity declining parameter. Otherwise, acid fracturing should be used. The reason is acid fracturing itself cannot usually provide enough fracture conductivity for improving production performance.

### Computer Implementation

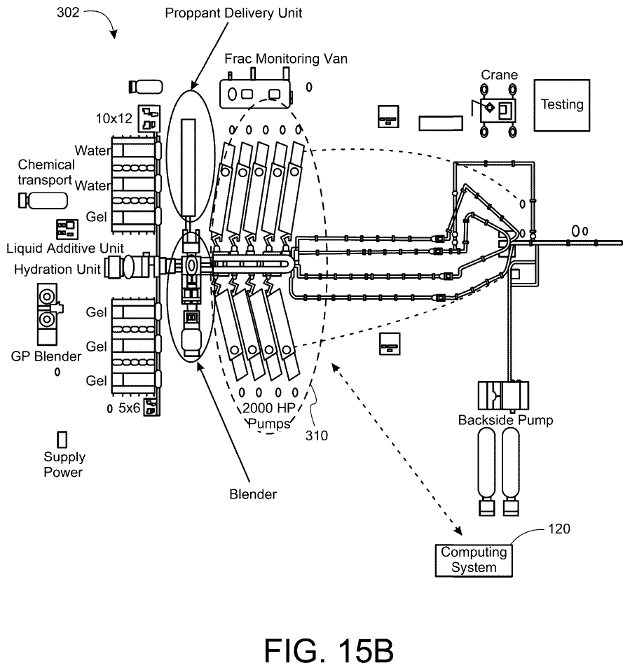

FIG. 14 is a flowchart of an example method 250 for determining fracturing methods for a wellbore. The computing system 120 implements the method 250 to determine the fracturing method for one or more depth intervals of the wellbore 102.

At block 252, depth-specific data is collected from the wellbore 102. In some examples, the data is acquired from well logs (e.g., from logging device 112), well drilling reports, well trajectory, formation tops, and/or borehole image logs.

At block 254, the computing system 120 determines a rock type of the formation based on the rock quality (e.g., porosity) and lithology data from the depth-specific data. In some examples, the computing system 120 determines the rock type as a range between 1 and 6 for a plurality of depths as described with reference to FIG. 3.

At block 256, the computing system 120 identifies sweet spot locations based on the rock type. For example, the computing system 120 identifies depth intervals of the wellbore 102 that have rock types above a threshold (e.g., above 4) and associates these depth intervals as being best for sweet spots. In some examples, the computing system 120 identifies the sweet spots as a function of depth as described with reference to FIG. 3.

At block 258, the computing system 120 generates a 1 dimensional (1D) mechanical earth model of the wellbore and formation. In some examples, the computing system 120 implements the processes described in U.S. Pat. No. 11,391,135 to calculate (or generate) the mechanical earth model.

At block 260, the computing system 120 uses borehole image logs to determine the direction of maximum horizontal stress in the formation.

At block 262, the computing system 120 calibrates the in-situ stress of the formation based on the drilling mud weight and borehole image log data. In some examples, the computing system 120 calibrates the mechanical earth model based on the drilling mud weight and borehole image log data to improve the accuracy of the mechanical earth model. In some examples, the computing system 120 implements the process described under the Breakdown Pressure and Perforation Direction heading to calibrate the in-situ stress of the formation 104.

At block 264, the computing system 120 calculates the breakdown pressure envelope, optimal perforation directions, acid fracture conductivity parameter, and fracture conductivity declining parameter for a plurality of depth intervals of the wellbore 102. In some examples, the computing system 120 implements the process described under the Breakdown Pressure and Perforation Direction heading calculate the breakdown pressure envelope, optimal perforation directions, acid fracture conductivity parameter, and fracture conductivity declining parameter for a plurality of depth intervals of the wellbore 102.

In some examples, the computing system 120 implements the processes described under the Breakdown Pressure and Perforation Direction heading above to determine the breakdown pressure envelopes and optimal perforation directions for a plurality of depth intervals of the wellbore 102. In some examples, the computing system 120 determines the acid fracture conductivity parameter by evaluating Eq. 58. In some examples, the computing system 120 determines the fracture conductivity declining parameter by evaluating Eq. 59.

At block 266, the computing system 120 determines potential perforation zones where rock quality is good (e.g., rock type above a threshold (e.g., above 4)) and the breakdown pressure are sufficiently low (e.g., below a threshold (e.g., below an average breakdown pressure)) for easy fracture initiation.

In the example wellbore system 100, the perforations are already formed in the representation shown in FIG. 1. However, in some implementations, the computing system 120 determines the locations of the perforation intervals before they are formed and controls or influences the decision-making process to determine where to form the perforation intervals. Each potential perforation interval is associated with a depth interval of the wellbore 102. In some examples, the computing system 120 determines stages 106A, 106B, and 106C as shown in FIG. 1 as the desired perforation zones. Each stage 106A-C is associated with a respective depth interval 108AC as described with reference to FIG. 1.

At block 268, for each potential perforation zone and associated depth interval, the computing system 120 determines whether the required breakdown pressure for clustered-perforation hydraulic fracturing exceeds the completion limits of the well 101. If so, the method proceeds to block 270, and determines to use oriented perforations. Otherwise, the method proceeds to block 272, and determines to use conventional perforations.

At block 274, for each potential perforation zone and associated depth interval, the computing system 120 determines whether the acid fracture conductivity parameter is higher than a first threshold (e.g., 0.15) and whether the fracture conductivity decline parameter is below a second threshold (e.g., 0.5).

If the acid fracture conductivity parameter is higher than the first threshold and the fracture conductivity decline parameter is below the second threshold, the method proceeds to block 276, and determines to use acid fracturing at the particular depth interval of the wellbore 102. Otherwise, the method proceeds to block 278, and determines to use proppant fracturing at the particular depth interval of the wellbore 102.

At block 280, the computing system 120 verifies the selected fracturing method using hydraulic fracturing modeling. In some examples, the computing system 120 (e.g., with the assistance of an engineer) solves a numerical model to confirm that the perforation tunnels would be sufficiently formed in the formation and would be sufficiently stimulated using the determined perforation locations and fracturing method. In some examples, the selected fracturing method verifies the fracture conductivity results. In some examples, the main pump schedule is optimized through 3D hydraulic fracturing modeling. It is important to estimate the actual effective conductivity derived from baseline conductivity.

Single Well Example with Proppant Fracturing and Acid Fracturing

FIGS. 15A-15C are diagrams of an example wellbore system 300 where both proppant fracturing and acid fracturing are used in a single well. While shown separately from the wellbore system 100 of FIG. 1, in some examples, wellbore system 300 is the same, or substantially the same, as wellbore system 100. As shown in FIG. 15B, wellbore system 300 includes a first fracturing fluid system and equipment set up 302 (e.g., a proppant fracturing). As shown in FIG. 15C, the wellbore system 300 includes a second fracturing fluid system and equipment set up 304 (e.g., an acid fracturing). In these examples, the only difference in set up 302 compared to set up 304 is additional equipment such as the proppant delivery unit and blender. Based on the computing system 120 output for treatment type, decision is made to switch from proppant fracturing to acid fracturing. A series of pumps 310 delivers the fracturing fluid into the wellbore 308 to stimulate the formation based on the fracturing method determination of blocks 274, 276, and 278. The wellhead includes a wing valve and a kill line.

In the example shown in FIG. 15, the computing system 120 determines to use proppant fracturing in the first, third, and fifth stages of the wellbore 308 and acid fracturing in the second and fourth stages of the wellbore 308. Each stage is fluidly isolated from each other using frac plugs to minimize cross-flow of the fracturing fluids between different stages. In some examples, the frac plugs are removed before the well is put into production. In some examples, the frac plugs remain in the wellbore 308 and allow upstream flow through hollow or dissolvable frac plugs but restrict downstream flow.

### Example Demonstration

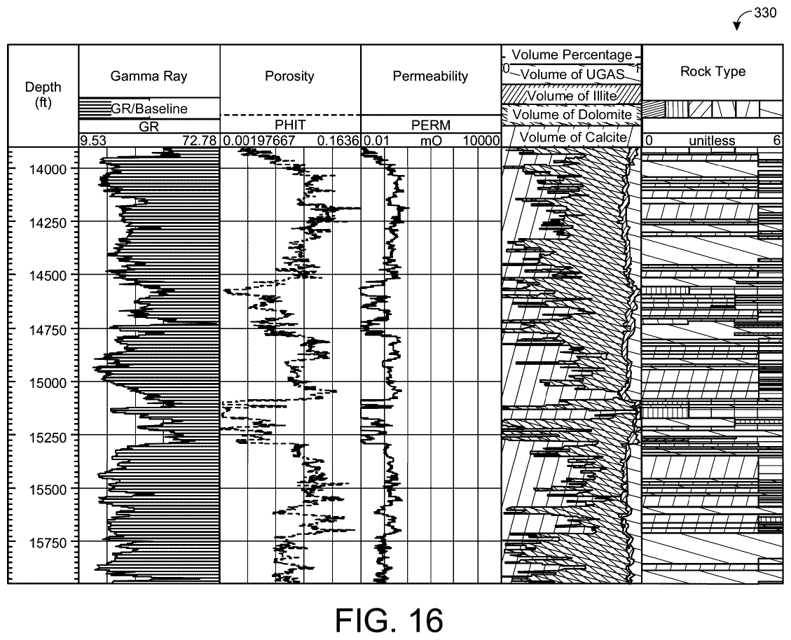

FIG. 16 is an example log plot 330 for rock type and sweet spot identification. Track 1 (from left) indicates the measured depth of the landing part in pay zone. Track 2 indicates gamma ray information of the formation which represents the natural radioactivity of earth materials in a wellbore. Track 3 indicates the porosity of the formation. Track 4 represents the permeability of the formation. Track 5 indicates the volume fraction of minerals of the formation (UGAS (Gas in unflushed zone), illite, calcite, and dolomite). Track 6 indicates the determined rock type. The computing system 120 determines the rock type based on the lithology and porosity of the formation. The rock type is indicative of rock quality in carbonate formations. Higher rock types represent better reservoir quality and are indicative of sweet spots in the formation for optimal perforation locations.

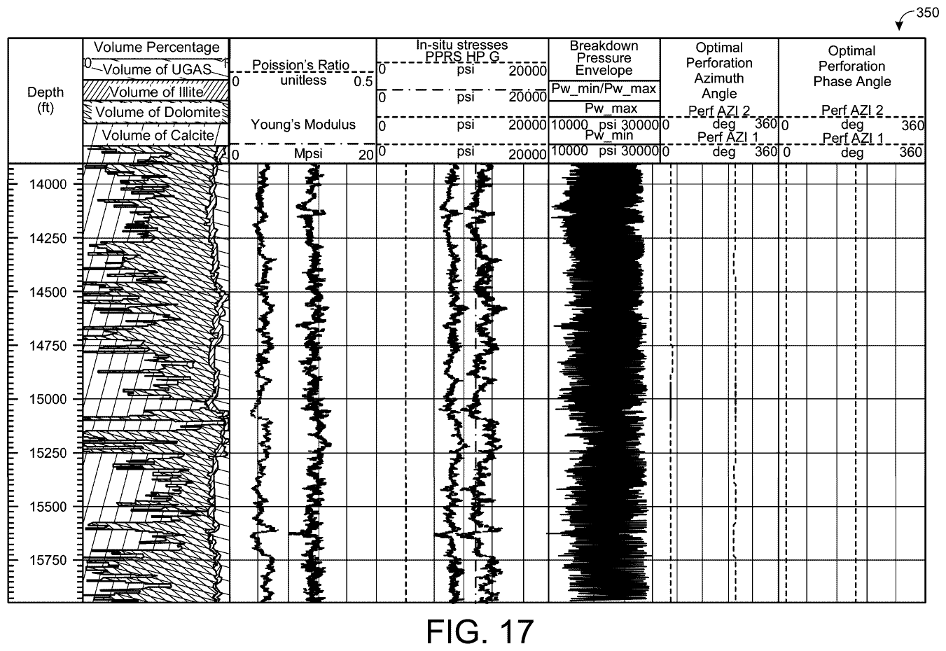

FIG. 17 is an example log plot 350 that shows further data and quantities of the same well as FIG. 16 for the same depths shown in FIG. 16. Track 1 indicates measured depth. Track 2 indicates the volume fraction of minerals and is the same as FIG. 16. Track 3 indicates rock properties (Poisson's ratio and Youngs modulus). Track 4 indicates in-situ stresses (hydrostatic pressure, vertical external stress, minimum horizontal stress, and maximum horizontal stress). Track 5 indicates breakdown pressure envelopes. Tracks 6 and 7 indicate optimal perforation directions (azimuth angle and phase angle, respectively).

As shown in FIG. 17 depth intervals with high volume fraction of dolomite are associated with high Young's modulus and high in-situ stresses. The in-situ stresses are calibrated by the computing system 120 based on caliper log data and drilling mud weight being used. In some examples, the caliper log data is acquired by logging device 112 and provides a continuous measurement of the size and shape of a borehole along its depth.

Track 5 indicates the minimum and maximum breakdown pressures along the well trajectory. Minimum breakdown pressure requires the perforations to be shot at the optimal perforation direction shown in Tracks 6 and 7. This means, for example, a 2-ft perforation depth interval should use a conventional perforation strategy with a 60° phase angle. In practice, only few perforations might be shot close to the optimal perforation direction if an oriented perforation technique is not used. In this situation, the breakdown pressure might be well above the minimum breakdown pressure. For deep and tight gas reservoirs, this can cause breakdown issues and lead to a failure to execute the fracturing job. For a well, the maximum and minimum breakdown pressures vary greatly with depth and are dependent on the well trajectory, in-situ stresses, etc., as shown in FIG. 17.

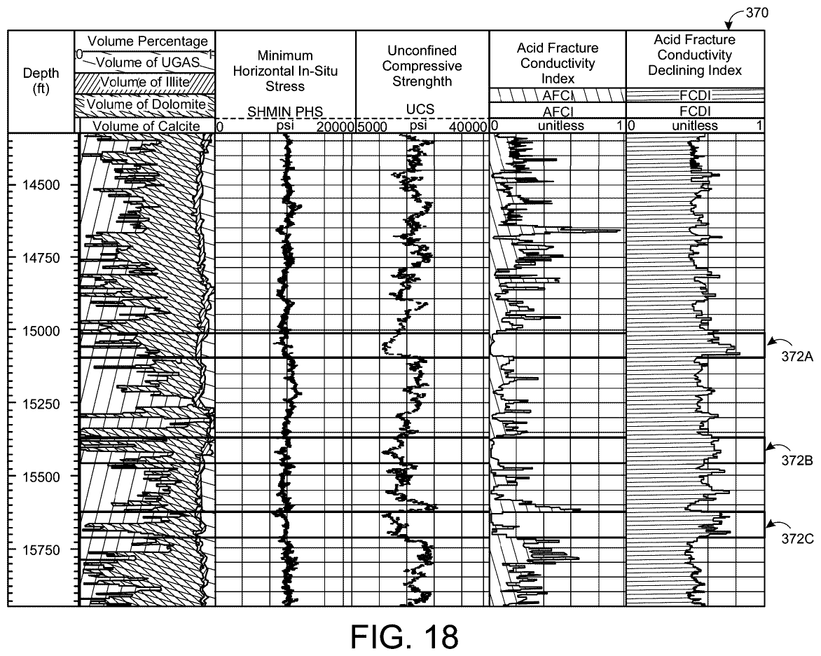

FIG. 18 is an example log plot 370 that shows further data and quantities of the same well as FIGS. 16 and 17 for the same depths shown in FIGS. 16 and 17. Track 1 indicates measured depth. Track 2 indicates the volume fraction of minerals (same as FIGS. 16 and 17). Track 3 indicates the minimum horizontal stress (also shown in FIG. 16). Track 4 indicates the unconfined compressive strength (UCS) of the formation. Track 5 indicates the acid fracture conductivity parameter of the formation. Track 6 indicates the fracture conductivity declining perimeter of the formation.

The acid fracture conductivity parameter is calculated using the acid fracture conductivity with acid fracture width wᵢ set equal to 0.2. A high acid fracture conductivity parameter formation indicates better fracturing performance while a lower acid fracture conductivity parameter can imply the formations are not suitable for acid fracturing. A high fracture conductivity declining parameter means that the uneven fracture surface might be crushed under the formation closure pressure, where the acid fracture conductivity is difficult to be maintained.

The computing system 120 determines zones 372A, 372B, and 372C as having lower acid fracture conductivity parameters (e.g., below a first threshold (e.g., between 0 and 0.1, or 0.02, 0.04, 0.06)) and high fracture conductivity declining parameter (e.g., above a second threshold (e.g., between 0.5 and 1.0, or 0.6, 0.8). In these zones, acid fracturing is not a good choice and proppant fracturing should be used for better production performance. In some examples, the computing system 120 uses proppant fracturing to fracture the formation in zones 372A-C.

Each vertical line in the plot represents an increase of 0.2 in the acid fracture conductivity and fracture conductivity declining parameters. In some examples, outliers and/or narrow spikes are filtered out of the acid fracture conductivity and fracture conductivity declining parameters (e.g., by averaging of the data as a function of depth).

After calculating all parameters related to rock type, acid fracture conductivity, breakdown pressure, and optimal perforation directions along the targeted pay zones, the computing system 120 determines the suggested perforation locations for clustered perforations and whether acid fracturing or proppant fracturing is preferred. For fracturing deep and tight carbonate gas reservoirs, the wellbore should be perforated where rock quality is good while ensuring that breakdown pressure is relatively low.

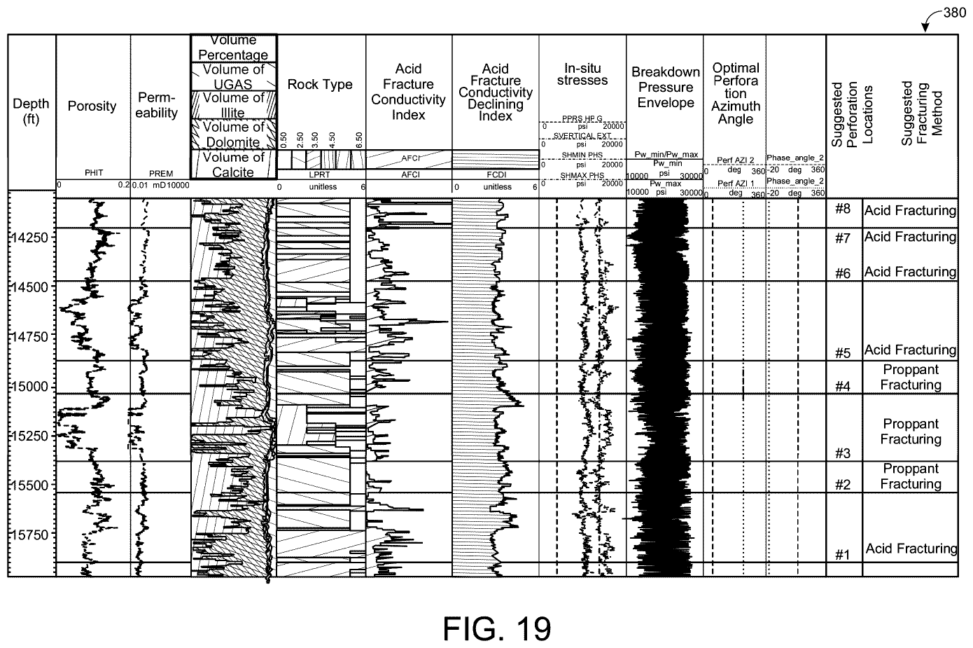

FIG. 19 is an example log plot 380 that shows data of FIGS. 16-18 together and specifies the suggested perforation locations and suggested fracturing method (e.g., acid fracturing vs. proppant fracturing). Track 1 indicates measured depth. Track 2 indicates the volume fraction of minerals (same as FIGS. 16-18). Track 3 indicates the rock type (same as FIG. 16). Track 4 indicates the acid fracture conductivity parameter (also shown in FIG. 18). Track 5 indicates the fracture conductivity declining parameter (also shown in FIG. 18). Track 6 indicates the in-situ stresses (also shown in FIG. 19). Track 7 indicates the breakdown pressure envelope (also shown in FIG. 19). Track 8 indicates the optimal perforation azimuth angle (also shown in FIG. 19). Track 9 indicates the optimal perforation phase angle (also shown in FIG. 19). Track 10 indicates the suggested perforation locations numbered by stage of the wellbore. Track 11 indicates the suggested fracturing methods.

The perforation clusters are numbers sequentially from the bottom. For perforation clusters 2-4, the rock types are good and the required breakdown pressure is lower than average. However, acid fracturing is unlikely to have good fracture conductivity (a lower acid fracture conductivity parameter and a higher fracture conductivity declining parameter). In this situation, proppant fracturing should be used instead of using acid fracturing.

For some segments, the rock qualities are good, but the required breakdown pressures may be higher than that wellhead can safely provide. In such cases, oriented perforations are preferred. Tracks 10 and 11 indicate the optimal perforation directions (perforation azimuth and perforation phase angle) at each measured depth, along which the fractures can be initiated relatively easier compared to the other perforation directions.

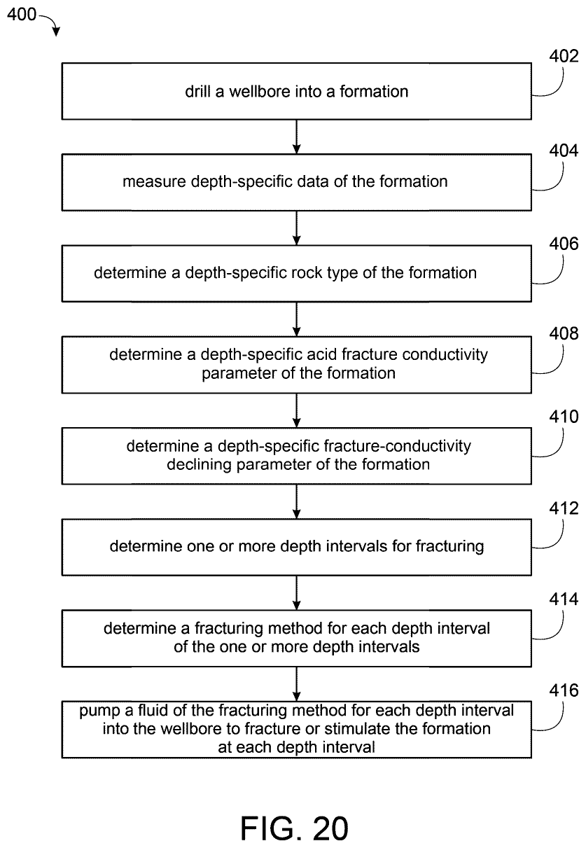

FIG. 20 is a flowchart of an example method 400 for fracturing the wellbore 102. In some implementations, all steps of the method 400 are controlled or performed by the computing system 120.

At step 402, a wellbore is drilled into a formation. For example, the wellbore 102 is drilled into the formation 104 by a drilling rig.

At step 404, depth-specific data of the formation is measured using a logging device. For example, data of the formation 104 is measured by the logging device 112 and expressed as a function of well trajectory. In some examples, the data includes porosity, density, permeability, lithology information, and/or geometry information (e.g., borehole size information) of the wellbore 102 or formation 104. In some examples, the lithology information includes a volume of anhydrite, a volume of calcite, and/or a volume of dolomite. The computing system 120 receives the data from the logging device 112. In some examples, the data of the formation 104 is measured by the logging device 112 is measured after a drilling phase of the well and before the completion of the well.

At step 406, a depth-specific rock type of the formation is determined. For example, the computing system 120 determines a rock type representing a quantitative indication of a quality of the formation for sweet spot identification and fracturing. In some examples, the computing system 120 determines the rock type based on porosity and lithology information of the formation as described with reference to Table 1 and FIG. 16.

At step 408, a depth-specific acid fracture conductivity parameter of the formation is determined. For example, the computing system 120 determines an acid fracture conductivity parameter of the formation 104. In some examples, the computing system 120 determines the acid fracture conductivity parameter by evaluating Eq. 58.

At step 410, a depth-specific fracture conductivity declining parameter of the formation is determined. For example, the computing system 120 determines a fracture conductivity declining parameter of the formation 104. In some examples, the computing system 120 determines the fracture conductivity declining parameter by evaluating Eq. 59. In some examples, the computing system 120 determines the fracture conductivity declining parameter based on a fracture closure pressure of the formation.

At step 412, one or more depth intervals for fracturing the formation are determined. For example, the computing system 120 determines one or more depth intervals (e.g., intervals 372A-C) for fracturing the formation based on a depth-specific breakdown pressure envelope of the formation, the rock type of the formation, the acid fracture conductivity parameter of the formation, and the fracture conductivity declining parameter of the formation.

At step 414, a fracturing method for each depth interval of the one or more depth intervals is determined. For example, the computing system 120 determines the fracture method as either acid fracturing or proppant fracturing for each depth interval based on the depth-specific acid fracture conductivity parameter of the formation and the depth-specific fracture conductivity declining parameter of the formation.

At step 416, a fluid of the fracturing method is pumped into the wellbore for each depth interval to stimulate the formation at each depth interval. For example, an acid fracturing fluid is pumped into the wellbore for some depth intervals and a proppant fracturing fluid is pumped into the wellbore for other depth intervals.

In some examples, a pump schedule is determined and the fluid is pumped based on the pump schedule. For example, the fracturing method is determined to be acid fracturing or a proppant fracturing and the system determines a pump schedule based on the fracturing method such that (i) when acid fracturing is determined as the fracturing method, the pump schedule is determined to include injecting fluid without a proppant, and (ii) when proppant fracturing is determined as the fracturing method, the pump schedule is determined to include injecting slurry that includes a proppant.

FIG. 21 is a schematic illustration of an example controller 500 (or control system) for determining the fracturing treatment for particular depth intervals of the wellbore according to the present disclosure. For example, the controller 500 may include or be part of the computing system 120. The controller 500 is intended to include various forms of digital computers, such as printed circuit boards (PCB), processors, digital circuitry, or otherwise parts of a system for determining a subsurface formation breakdown pressure. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 500 includes a processor 510, a memory 520, a storage device 530, and an input/output device 540 (for displays, input devices, example, sensors, valves, pumps). Each of the components 510, 520, 530, and 540 are interconnected using a system bus 550. The processor 510 is capable of processing instructions for execution within the controller 500. The processor may be designed using any of a number of architectures. For example, the processor 510 may be a CISC (Complex Instruction Set Computers) processor, a RISC (Reduced Instruction Set Computer) processor, or a MISC (Minimal Instruction Set Computer) processor.

In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the controller 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the controller 500. In one implementation, the storage device 530 is a computer-readable medium. In various different implementations, the storage device 530 may be a floppy disk device, a hard disk device, an optical disk device, or a tape device.

The input/output device 540 provides input/output operations for the controller 500. In one implementation, the input/output device 540 includes a keyboard and/or pointing device. In another implementation, the input/output device 540 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations have been described. Nevertheless, it will be understood that various modifications may be made without departing from the spirit and scope of the disclosure. For example, example operations, methods, or processes described herein may include more steps or fewer steps than those described. Further, the steps in such example operations, methods, or processes may be performed in different successions than that described or illustrated in the figures. Accordingly, other implementations are within the scope of the following claims.

### EXAMPLES

In some implementations, methods for fracturing a formation include: measuring, by a logging device, depth-specific data of the formation, the depth-specific data comprising porosity and lithology information of the formation; determining, by a computer, a depth-specific rock type of the formation based on the porosity and lithology information of the formation; determining, by the computer, a depth-specific acid fracture conductivity parameter of the formation; determining, by the computer, a depth-specific fracture conductivity declining parameter of the formation; determining, by the computer, one or more depth intervals for fracturing the formation based on the porosity and lithology information of the formation; determining, by the computer, a fracturing method for each depth interval of the one or more depth intervals based on the depth-specific acid fracture conductivity parameter of the formation and the depth-specific fracture conductivity declining parameter of the formation; and pumping, by a pump, a fluid of the fracturing method for each depth interval into the formation to fracture or stimulate the formation at each depth interval.

In an example implementation combinable with any other example implementation, the depth-specific rock type is determined based on a volume of calcite of the formation, a volume of dolomite of the formation, and a volume of anhydrite of the formation.

In an example implementation combinable with any other example implementation, the depth-specific rock type is a quantitative indication of a quality of the formation for sweet spot identification and fracturing.

In an example implementation combinable with any other example implementation, the depth-specific acid fracture conductivity parameter of the formation is determined based on a fracture closure pressure of the formation.

In an example implementation combinable with any other example implementation, the depth-specific acid fracture conductivity parameter of the formation is determined based on a rock embedment strength of the formation.

In an example implementation combinable with any other example implementation, the depth-specific acid fracture conductivity parameter of the formation is determined by evaluating the following equation: AFCI=4.3063×10⁴ e^{-C}^{₂}^{σ}^{_{c}}, where AFCI is the acid fracture conductivity parameter along a well trajectory of a well in a landing zone of the well in the formation, C₂=(13.9−1.3lnSₑₘ)×10⁻³ for Sₑₘ<20000 psi; C₂=(3.8−0.28lnSₑₘ)×10⁻³ for Sₑₘ>20000 psi; and σ_{c} is the fracture closure pressure in pounds per square inch (psi); and Sₑₘ is the rock embedment strength of the formation in psi.

In an example implementation combinable with any other example implementation, the method further includes: extracting a core plug from the formation; determining the rock embedment strength of the formation by performing laboratory testing of the core plug of the formation; and determining the fracture closure pressure of the formation by generating a 1-dimensional mechanical Earth model along the a well trajectory of a well, the 1-dimensional mechanical Earth model representing the formation.

In an example implementation combinable with any other example implementation, the depth-specific fracture conductivity declining parameter is determined based on a fracture closure pressure of the formation.

In an example implementation combinable with any other example implementation, the depth-specific fracture conductivity declining parameter of the formation is determined based on an unconfined compressive strength of the formation.

In an example implementation combinable with any other example implementation, the depth-specific fracture conductivity declining parameter of the formation is determined by evaluating the following equation:

$$= \frac{{σ}_{c}}{U C S} ,$$

where FCDI is the depth-specific fracture conductivity declining parameter, oc is the fracture closure pressure in pounds per square inch (psi), and UCS is the unconfined compressive strength of the formation in psi.

In an example implementation combinable with any other example implementation, the fracturing method is determined to be acid fracturing or a proppant fracturing.

In an example implementation combinable with any other example implementation, the method comprises determining a pump schedule based on the fracturing method such that (i) when acid fracturing is determined as the fracturing method, the pump schedule is determined to include injecting fluid without a proppant, and (ii) when proppant fracturing is determined as the fracturing method, the pump schedule is determined to include injecting slurry that includes a proppant.

In an example implementation combinable with any other example implementation, determining the fracturing method for each depth interval comprises determining the fracturing method for a first depth interval as acid fracturing and determining the fracturing method for a second depth interval as proppant fracturing.

In an example implementation combinable with any other example implementation, determining the fracturing method for each depth interval comprises determining that the fracturing method is proppant fracturing when (i) the depth-specific acid fracture conductivity parameter is below a first threshold and (ii) the depth-specific fracture conductivity declining parameter is above a second threshold.

In an example implementation combinable with any other example implementation, determining the fracturing method for each depth interval comprises determining that the fracture method is acid fracturing when (i) the depth-specific acid fracture conductivity parameter is equal to or above the first threshold and (ii) the depth-specific fracture conductivity declining parameter is equal to or below the second threshold.

In an example implementation combinable with any other example implementation, the first threshold is between 0 and 0.2 and the second threshold is between 0.5 and 1.

In an example implementation combinable with any other example implementation the method further includes performing a numerical simulation to verify the fracture method for the formation at each depth interval.

In an example implementation combinable with any other example implementation, the one or more depth intervals are determined based on a depth-specific breakdown pressure envelope of the formation.

In an example implementation combinable with any other example implementation, the one or more depth intervals are determined based on the depth-specific acid fracture conductivity parameter of the formation and the depth-specific fracture conductivity declining parameter of the formation.

In an example implementation combinable with any other example implementation, pumping the fluid of the fracturing method for each depth interval into the formation comprises pumping an acid into the formation when the fracturing method is determined to be acid fracturing.

In an example implementation combinable with any other example implementation, pumping the fluid of the fracturing method for each depth interval into the formation comprises pumping a proppant into the formation when the fracturing method is determined to be proppant fracturing.

What is claimed is:

1. A method for fracturing a formation, the method comprising:
measuring, by a logging device, depth-specific data of the formation, the depth-specific data comprising porosity and lithology information of the formation;
determining, by a computer, a depth-specific rock type of the formation based on the porosity and lithology information of the formation;
determining, by the computer, a depth-specific acid fracture conductivity parameter of the formation;
determining, by the computer, a depth-specific fracture conductivity declining parameter of the formation;
determining, by the computer, one or more depth intervals for fracturing the formation based on the porosity and lithology information of the formation;
determining, by the computer, a fracturing method for each depth interval of the one or more depth intervals based on the depth-specific acid fracture conductivity parameter of the formation and the depth-specific fracture conductivity declining parameter of the formation; and
pumping, by a pump, a fluid of the fracturing method for each depth interval into the formation to fracture or stimulate the formation at each depth interval.

2. The method of claim 1, wherein the depth-specific rock type is determined based on a volume of calcite of the formation, a volume of dolomite of the formation, and a volume of anhydrite of the formation.

3. The method of claim 2, wherein the depth-specific rock type is a quantitative indication of a quality of the formation for sweet spot identification and fracturing.

4. The method of claim 1, wherein the depth-specific acid fracture conductivity parameter of the formation is determined based on a fracture closure pressure of the formation.

5. The method of claim 4, wherein the depth-specific acid fracture conductivity parameter of the formation is determined based on a rock embedment strength of the formation.

6. The method of claim 5, wherein the depth-specific acid fracture conductivity parameter of the formation is determined by evaluating the following equation:
$$AFCI = 4.3063 \times 1 {0}^{4} {e}^{- {C}_{2} {σ}_{c}} ,$$
where AFCI is the acid fracture conductivity parameter along a well trajectory of a well in a landing zone of the well in the formation, C₂=(13.9−1.3lnSₑₘ)×10⁻³ for Sₑₘ<20000 psi; C₂=(3.8−0.28lnSₑₘ)×10⁻³ for Sₑₘ>20000 psi; and σ_{c} is the fracture closure pressure in pounds per square inch (psi); and Sₑₘ is the rock embedment strength of the formation in psi.

7. The method of claim 5, further comprising:
extracting a core plug from the formation;
determining the rock embedment strength of the formation by performing laboratory testing of the core plug of the formation; and
determining the fracture closure pressure of the formation by generating a 1-dimensional mechanical Earth model along a well trajectory of a well, the 1-dimensional mechanical Earth model representing the formation.

8. The method of claim 1, wherein the depth-specific fracture conductivity declining parameter is determined based on a fracture closure pressure of the formation.

9. The method of claim 8, wherein the depth-specific fracture conductivity declining parameter of the formation is determined based on an unconfined compressive strength of the formation.

10. The method of claim 9, wherein the depth-specific fracture conductivity declining parameter of the formation is determined by evaluating the following equation:
$$FCDI = \frac{{σ}_{c}}{UCS} ,$$
where FCDI is the depth-specific fracture conductivity declining parameter, σ_{c} is the fracture closure pressure in pounds per square inch (psi), and UCS is the unconfined compressive strength of the formation in psi.

11. The method of claim 1, wherein the fracturing method is determined to be acid fracturing or a proppant fracturing.

12. The method of claim 11, further comprising determining a pump schedule based on the fracturing method such that (i) when acid fracturing is determined as the fracturing method, the pump schedule is determined to include injecting fluid without a proppant, and (ii) when proppant fracturing is determined as the fracturing method, the pump schedule is determined to include injecting slurry that includes a proppant.

13. The method of claim 11, wherein determining the fracturing method for each depth interval comprises determining the fracturing method for a first depth interval as acid fracturing and determining the fracturing method for a second depth interval as proppant fracturing.

14. The method of claim 1, wherein determining the fracturing method for each depth interval comprises determining that the fracturing method is proppant fracturing when (i) the depth-specific acid fracture conductivity parameter is below a first threshold and (ii) the depth-specific fracture conductivity declining parameter is above a second threshold.

15. The method of claim 14, wherein determining the fracturing method for each depth interval comprises determining that the fracture method is acid fracturing when (i) the depth-specific acid fracture conductivity parameter is equal to or above the first threshold and (ii) the depth-specific fracture conductivity declining parameter is equal to or below the second threshold.

16. The method of claim 15, wherein the first threshold is between 0 and 0.2 and the second threshold is between 0.5 and 1.

17. The method of claim 1, wherein the one or more depth intervals are determined based on a depth-specific breakdown pressure envelope of the formation and the method further comprises performing a numerical simulation to verify the fracture method for the formation at each depth interval.

18. The method of claim 17, wherein the one or more depth intervals are determined based on the depth-specific acid fracture conductivity parameter of the formation and the depth-specific fracture conductivity declining parameter of the formation.

19. The method of claim 1, wherein pumping the fluid of the fracturing method for each depth interval into the formation comprises pumping an acid into the formation when the fracturing method is determined to be acid fracturing.

20. The method of claim 19, wherein pumping the fluid of the fracturing method for each depth interval into the formation comprises pumping a proppant into the formation when the fracturing method is determined to be proppant fracturing.
